(19) United States
(12) Patent Application Publication
(10) Pub. No.: US 2026/0234559 A1
(43) Pub. Date: Aug. 13, 2026

(54) ARMOURED REGULATORY T CELL

(71) Applicant: KING'S COLLEGE LONDON, London (GB)

(72) Inventors: Robert Page, London (GB); Sophie Papa, London (GB); Shahram Kordasti, London (GB); Rita Antunes Dos Reis, London (GB)

(21) Appl. No.: 19/153,221

(22) PCT Filed: Feb. 16, 2024
(86) PCT No.: PCT/EP2024/053973

§ 371 Date: Aug. 1, 2025

(30) Foreign Application Priority Data
Feb. 16, 2023 (GB) 2302212.2

(51) Int. Cl. C12N 5/0783; A61K 35/17; A61P 7/06; C07K 14/715; C07K 16/28; C12N 15/113

(52) U.S. Cl. CPC C12N 5/0637; A61K 35/17; A61P 7/06; C07K 14/7158; C07K 16/2896; C12N 15/1138; C07K 2317/53; C07K 2317/622; C07K 2319/03; C12N 2310/14; C12N 2310/141; C12N 2510/00

(57) ABSTRACT

The present invention relates to a regulatory T-cell (Treg) genetically engineered to express a non-activating site-targeting receptor or ligand and a reduced expression level of FAS. Also provided are polynucleotides, vectors, pharmaceutical compositions, methods of genetically engineering the Treg and methods of treatment or prevention of a disease.

### TECHNICAL FIELD

This invention relates to a regulatory T-cell (Treg) genetically engineered to express a non-activating site-targeting receptor or ligand and a reduced expression level of FAS. Also provided are polynucleotides, vectors, pharmaceutical compositions, methods of genetically engineering the Treg and methods of treatment or prevention of a disease.

### BACKGROUND

Aplastic anaemia (AA) is a serious blood condition characterized by failure of the bone marrow to produce blood cells. While there are various known causes of AA, one of the primary causes can be immune-mediated bone marrow failure (BMF), known as immune AA (iAA). This is characterized by an immunological imbalance comprising immune mediated destruction of bone marrow stem/progenitor cells and diminished anti-inflammatory regulatory T cells (Tregs). Previous studies have found that the number and subpopulations of Tregs inversely correlate with AA disease severity and response to treatment.

Current treatments for iAA include immune suppressive therapy (IST) and hematopoietic stem cell transplant (HSCT). However, these treatments have significant associated morbidity and mortality. Other treatments may include the administration of a thrombopoietin receptor agonist, the effect of which on immune imbalance on iAA being unclear. Although the addition of thrombopoietin receptor agonist (eltrombopag) to the standard IST has shown a high rate of complete response among subjects with severe AA, relapse rate and clonal evolution were comparable to standard IST therapy alone. Therefore, there remains a need for improved treatments for iAA, particularly for subjects who are not eligible for IST, HSCT, or subjects with non-severe disease.

Regulatory T cells (Tregs) are a non-redundant, suppressive population of CD4⁺ T cells which function to suppress inflammation. Tregs have been used therapeutically to treat various diseases and conditions, including inflammatory conditions such as autoimmune disease.

To date, however, the therapeutic utilization of Tregs has not reached its full potential. While Tregs can be expanded in vitro to form a stable and functional Treg population, this expansion is limited by low initial Treg numbers in some conditions, such as AA. This can make it challenging to expand Tregs to the cell numbers required for conventional cell therapy. In addition, Tregs can exhibit sensitivity to the inflammatory environment, which can reduce efficacy.

The present invention seeks to address one or more of the aforementioned issues.

### SUMMARY OF THE INVENTION

The present invention provides a regulatory T-cell (Treg) genetically engineered to express a) a non-activating site-targeting receptor or ligand and b) a reduced expression level of FAS.

Also provided is an or a plurality of polynucleotide sequence(s) comprising a) a nucleotide sequence which reduces the expression level of FAS and b) a nucleotide sequence encoding a non-activating site-targeting receptor or ligand. The a or plurality of polynucleotide sequence(s) may further comprise a nucleotide sequence encoding a chemokine receptor or functional variant thereof.

The invention also provides a vector comprising the polynucleotide sequence(s) of the invention.

There is also provided a method of modifying a Treg, the method comprising genetically engineering a Treg to express a) a non-activating site-targeting receptor or ligand; and b) a reduced expression level of FAS.

Further provided is a pharmaceutical composition comprising the Treg, polynucleotide sequence(s) and/or the vector of the present invention and a pharmaceutically or physiologically acceptable diluent and/or carrier.

There is also provided the Treg, polynucleotide sequence(s), vector or the pharmaceutical composition of the present invention for use in the treatment or prevention of a disease. Also provided is a method of treating or preventing a disease in a subject, wherein the method comprises administering to the subject the Treg, polynucleotide sequence(s), vector or the pharmaceutical composition of the present invention. In some embodiments the disease comprises an autoimmune disease. In some embodiments the disease comprises Aplastic Anaemia (AA).

### BRIEF DESCRIPTION OF THE DRAWINGS

One or more embodiments of the invention will now be described, by way of example only, with reference to the accompanying drawings, in which:

FIG. 1: (a) viSNE plots generated from CyTOF data (total CD4⁺ T cells on the left and overlay of Tregs on total CD4⁺ T cells on the right) revealed two subpopulations within Tregs, designated as Treg-A and -B (arrows). (b) Median expression of the eight most discriminative parameters between the two Treg subpopulations identified by the automated clustering algorithm FLOCK. Expression values were transformed using the asinh function in a cofactor of 5. Heat map plot is based on 19 aplastic anaemia (AA) samples and 5 healthy donor (HD) sample. (c) IPA analysis on the RNA sequencing data shows the ranking of top 10 canonical pathways by −log(P value) in AA Treg-B (n=3) compared with AA Treg-A (n=3). Significant upregulation of FAS in 3 out of 10 pathways is indicated. (d) The percentage of early (Viability Dye eFluor 780-Annexin V⁺) and late (Viability Dye eFluor 780+ Annexin V⁺) apoptotic cells in Treg-A and Treg-B before and after treatment with 5 μg/mL of anti-Fas. Error bars represent mean±standard deviation (SD) 7. **P≤0.01, ***P≤0.001.

FIG. 2: (a-b) Western blot analysis of STAT5 and pSTAT5 protein expression in Treg-A and Treg-B after treatment with 1, 40, 60, or 80 IU/ml of human IL-2 for 15 or 30 minutes. β-ACTIN protein level is used as a loading control, and numbers represent the densitometric quantification of STAT5 and pSTAT5 protein expression levels normalized to β-ACTIN7. (c) The expansion rate of Treg-A and Treg-B from HD (n=6) and AA subjects (n=3). Treg-A and Treg-B were stimulated with anti-CD3/CD28 beads (1 cell: 1 bead ratio) and 1000 IU/mL IL-2 for 4 weeks with 2 μM ATRA and 100 nM rapamycin in T-cell expansion medium. Error bars represent mean±SD. *P≤0.05; ns, not significant. (d) The percentage of proliferation of CFSE-stained Tcon cells when cocultured with autologous HD and AA-expanded total Tregs in 8:1, 4:1, 2:1, and 1:1 Tcon:Treg ratios for 5 days in the presence of anti-CD3/CD28 beads (Treg:beads=20:1).

FIG. 3: (a) Luciferase-GFP transduced Tregs (t-Tregs) were expanded for 4 weeks and injected to NSG-SGM3 humanized mice on their own (I), in 1:1 ratio with PBMCs (II). The control group were injected with PBMCs only (III). All groups were imaged weekly by whole-body bioluminescence imaging (IVIS), up to 6 weeks. (b) Overall survival of mice that were injected with AA subject derived PBMCs without (n=5) or with AA Tregs (autologous or allogenic) (n=8) or AA Tregs alone (n=2). Those mice in group I and II (injected with Tregs) survived significantly longer (p<0.001). (c) Representative histology tissue sections of the mouse femur bone. Mice that were injected with Tregs (I & II) show higher bone marrow cellularity compared to the control group (III, PBMCs only).

FIG. 4: Knockdown of FAS by miRshFAS: (a) FAS KD of proliferating Tregs by shRNA incorporated into miR30 (b) miRshFAS1 shows more than 90% efficacy to KD FAS (c) FAS⁻/low Tregs are FAS-L mediated apoptosis resistant (d) Modified Tregs can be expanded in vitro. (e) Modified Tregs remain functional in vitro.

FIG. 5: FAS MFI following knockdown by miRshFAS: (a) FAS MFI (CD95 MFI) is shown from Tregs from 3 healthy donors. The graph shows the MFI comparison between untransduced cells, miRshFAS1, miRshFAS2, miRshGFP (which has no target, works as a negative control) and miRshEmpty (which is a control vector without any miRshRNA). MFI was reduced by at least 50% in Tregs transduced with miRshFAS1 or miRshFAS2. (b) Stability of CD95 knockdown: MFI of FAS (CD95) 4 weeks and 6 weeks after Treg transduction with miRshFAS, miRshGFP and untransduced from three different donor cell populations.

FIG. 6: (a-b) The 34-m-scFv/Faskd construct for Fas KD and simultaneous expression of anti-CD34 was utilized to engineer mdTregs. Expression of anti-CD34 was confirmed by staining with mouse anti-myc (as the reporter) and secondary APC anti-mouse IgG. Additionally, recombinant Human CD34-Fc tagged fusion protein (cat 10103-H02H, Sino Biological) and secondary anti-Fc antibody (anti-human Alexa Fluor 647) were used for confirming the expression of functional anti-CD34 by mdTregs. Fas negative Tregs which were positive for both myc and CD34 binding are considered mdTregs (b).

FIG. 7: Knockdown of FAS by shRNA: Three different shRNA sequences to target FAS were tested, shFAS-1, shFAS-2 and shFAS-3. One shRNA was also designed to target GFP as a negative control with no target in the cell. TdTomato was used as a reporter gene to detect transduced Jurkat cells (a). shFAS-1 and shFAS-2 were particularly effective at knocking down FAS expression (b).

FIG. 8: FAS knockdown using a CRISPR system. (a) Percentage of FAS⁺ cells following transduction using two different guide RNAs targeting FAS. GFP was used as a reporter gene to detect transduced cells. Both guide RNAs reduced the proportion of FAS⁺ cells. (b) FAS MFI following transduction using two different guide RNAs targeting FAS in Tregs from a healthy donor. GFP was used as a reporter gene to detect transduced cells. MFI was also reduced by both guide RNAs.

FIG. 9: Expression of CXCR4 ligand and knockdown of FAS by miRshFAS1: (a) The CXCR4_miRshFAS1 construct for Fas KD or the negative control CXCR4_miRshGFP construct for GFP KD, both co-expressing CXCR4 were utilized to engineer mdJurkats. (b) Expression of CXCR4 was confirmed by staining with anti-human CD184 (CXCR4) antibody (cat 306518). (c) Representative FACS plot showing the simultaneous expression of CXCR4 and knockdown of FAS by miRshFAS1 and the negative control miRshGFP.

FIG. 10: (a-d) Different linkers were used to optimize the 34-m-scFv for both 34-m-scFv/Faskd and 34-m-scFv/GFPkd constructs. (a) Constructs were used to transfect HEK293 Ts cells and expression of aCD34 was confirmed by flow cytometry. (b) In Jurkat cells, the simultaneous expression of aCD34 and myc-tag was confirmed. As well as for the (c) the reporter gene TdTomato and aCD34. The cloning constructs were also used to modify Tregs (d) expression of TdTomato and aCD34 was confirmed by flow cytometry.

### DETAILED DESCRIPTION

The present invention provides a regulatory T-cell (Treg) genetically engineered to express a) a non-activating site-targeting receptor or ligand and b) a reduced expression level of FAS. As the skilled person will appreciate, FAS is the FAS receptor, which may otherwise be referred to as cluster of differentiation 95 (CD95), FASR, apoptosis antigen 1 (APO-1 or APT) or tumour necrosis factor receptor superfamily member 6 (TNFRSF6). FAS is a death receptor which is typically expressed at the cell membrane. When FAS specifically binds to its ligand, FAS ligand (FASL), this can lead to programmed cell death (apoptosis) of the FAS-expressing cell.

By “site-targeting receptor or ligand”, this will be understood to refer to a receptor or ligand which is capable of specifically binding to its target, the target being associated with/localised to the particular target site/anatomical location. The target is typically a cognate ligand (for a site-targeting receptor) or a cognate receptor (for a site-targeting ligand). Thus, by “site targeting”, this will be understood to mean that the non-activating site-targeting receptor or ligand is specific for a particular site/anatomical location. Preferably, expression of the non-activating site-targeting receptor or ligand by the Treg “homes” the Treg to the target site once introduced into a subject.

“Specifically binding” will be understood to mean that the non-activating site-targeting receptor or ligand has preferential or high affinity for its target. The non-activating site-targeting receptor or ligand has high affinity for the target if it binds with a Kd of 1×10-6 M or less, more preferably 1×10-7 M or less, 5×10-8 M or less, more preferably 1×10-8 M or less, more preferably 5×10-9 M or less, or more preferably 7×10-9 M or less. A molecule or group binds with low affinity if it binds with a Kd of 1×10-6 M or more, more preferably 1×10-5 M or more, more preferably 1×10-4 M or more, more preferably 1×10-3 M or more, even more preferably 1×10-2 M or more.

In some embodiments, the non-activating site-targeting receptor or ligand does not bind or binds with only low affinity to other or different molecules to the target, such as other or different receptors and/or ligands. Preferably, the non-activating site-targeting receptor or ligand binds to the target with an affinity that is at least 10 times, such as at least 50, at least 100, at least 200, at least 300, at least 400, at least 500, at least 1000 or at least 10,000 times, greater than its affinity for other molecules.

Affinity can be measured using known binding assays, such as those that make use of fluorescence and radioisotopes. Competitive binding assays are also known in the art. The strength of binding between peptides or proteins and other proteins can be measured, for example, using a surface plasmon resonance biosensor-based assay, as described in Fan et al. Biochemistry and Biophysics Reports 9:51-60 (2017).

In the context of the present invention, “non activating” defines a receptor or ligand which, upon specifically binding its target, does not activate or has reduced activation of the target at the target site. In the context of the present invention, activation will be considered reduced if the level of activation upon specific binding of the non-activating site-targeting receptor or ligand to its target is reduced relative to the level of activation following specific binding of the target by an activating site-targeting receptor or ligand or other receptor or ligand.

The non-activating site-targeting receptor or ligand may otherwise be referred to as a neutralising site-targeting receptor or ligand. As the skilled person will be aware, the term “neutralising” is used in the art to define a receptor or ligand which is capable, upon specific binding of its target, to neutralise the intracellular signalling activity of its target. Thus, the non-activating site-targeting receptor or ligand of the present invention is capable of reducing the intracellular signalling activity of its cognate receptor or ligand upon specific binding to the cognate receptor or ligand.

In addition to being neutralising, the non-activating site-targeting receptor or ligand may be a blocking site-targeting receptor or ligand. In the context of the present invention, a blocking site-targeting receptor or ligand will be understood to specify a receptor or ligand which, upon specifically binding its target, prevents or reduces the ability of the target from specifically binding to another molecule.

Activation of the target will typically be measured as intracellular signalling activity downstream of the target. In some embodiments, the non-activating site-targeting receptor or ligand is capable of reducing the intracellular signalling activity of its target by at least about 40%, at least about 50%, at least about 60%, at least about 70%, at least about 80%, at least about 90%, at least about 95%, or at least about 99%. In some embodiments, the non-activating site-targeting receptor or ligand is capable of reducing the intracellular signalling activity of its target by at least about 50%. In some embodiments, the non-activating site-targeting receptor or ligand is capable of reducing the intracellular signalling activity of its target by at least about 70%, optionally by at least about 80%. In some embodiments, the non-activating site-targeting receptor or ligand is capable of reducing the intracellular signalling activity of its target by of from about 50% to about 99%. In some embodiments, the non-activating site-targeting receptor or ligand is capable of reducing the intracellular signalling activity of its target by of from about 60% to about 99%. In some embodiments, the non-activating site-targeting receptor or ligand may reduce the intracellular signalling activity of its target such that the intracellular signalling activity of the target is undetectable.

Intracellular signalling activity of the target may comprise the transcription and/or translation of one or more activation genes, for example cytokines, transcription factors, chemokines, antibodies and so on in the cell in/on which the target is expressed. The skilled person will be aware of intracellular signalling activity of the target, and so will be able to select an appropriate method to detect the level of intracellular signalling activity. For example, the intracellular signalling activity may lead to the translation of one or more cytokines, transcription factors, chemokines or antibodies. In such instances, the skilled person may use ELISA, flow cytometry and/or fluorescence microscopy to detect the level of expression of the cytokines, transcription factors, chemokines or antibodies to determine a reduction in the intracellular signalling activity. In some instances, the intracellular signalling activity may lead to the transcription of one or more cytokine transcription factor, chemokine or antibody genes. In such embodiments, it will be appreciated that a read out of intracellular signalling activity may comprise cytokine, transcription factor, chemokine and/or antibody mRNA.

Various methods to detect a level of mRNA and/or protein are known in the art. Exemplary methods for the detection of mRNA include, but are not limited to Northern Blots, nuclease protection assays (NPAs), in situ hybridisation and reverse transcription-polymerase chain reaction (RT-PCR). The mRNA may be detected using RT-PCR. Suitable methods for the detection of protein include, but are not limited to western blots, immunoprecipitation, flow cytometry and fluorescence microscopy.

Typically, the non-activating site-targeting receptor or ligand of the present invention is genetically modified to be non-activating. Thus, the non-activating site-targeting receptor or ligand of the present invention is non-naturally occurring and typically comprises a site-targeting receptor or ligand which has been genetically engineered to be non-activating.

Therefore, in the context of the present invention, the non-activating site-targeting receptor or ligand is exogenous to the Treg cell. As the skilled person will appreciate, the term “exogenous” means that the non-activating site-targeting receptor or ligand is not native to the Treg cell; it has been introduced by genetic engineering.

The inventors have advantageously found that genetically engineering Tregs to express a) a non-activating site-targeting receptor or ligand and b) a reduced expression level of FAS means that fewer Tregs are needed than has traditionally been required for cell therapy. In particular, the reduced expression level of FAS increases the resistance of the genetically engineered Tregs to apoptosis. The expression of the non-activating site-targeting receptor or ligand aids the targeted homing of the genetically engineered Treg to a specific targeted site without activation or with reduced activation at the target site. This enables targeted homing of the genetically engineered Tregs to the target side while avoiding undesirable side effects. In addition, such genetic modification may facilitate a greater proportion of the original Treg population reaching the intended target site than for typical cell therapy. As such, fewer Tregs are required than for conventional cell therapy. Advantageously, this may improve the efficacy of therapy as well as the ease and cost of production of cells for cell therapy.

In some embodiments, the Tregs of the present invention are resistant to apoptosis.

As the skilled person will appreciate, a “Treg” may otherwise be referred to as a regulatory T-cell. Regulatory T-cells are an anti-inflammatory subset of CD4⁺ T-cells. Markers found on Tregs, and which can therefore be used to identify a Treg, will be known to the skilled person and are further discussed herein.

In the context of the present invention, “genetically engineered” means that the Treg cell has been genetically modified to express the non-activating site-targeting receptor or ligand and a reduced expression level of FAS. Typically, “genetically engineered” means that the nucleotide composition of the Treg cell has been altered to express a non-activating site-targeting receptor or ligand and a reduced expression level of FAS.

By “genetically engineered to express a non-activating site-targeting receptor or ligand”, this will be understood to refer to the Treg expressing a detectable level of the non-activating site-targeting receptor or ligand. Prior to genetic engineering, the Treg cell preferably does not express or expresses undetectable levels of the non-activating site-targeting receptor or ligand.

In the context of the present invention, “detectable level” may comprise a detectable level of mRNA and/or protein encoding, for example, the non-activating site-targeting receptor or ligand, or cytokine, transcription factor, chemokine and/or antibody mRNA as a readout of intracellular signalling activity. Preferably, “detectable level” comprises a detectable level of the non-activating site targeting receptor or ligand protein. “Detectable level” may comprise a detectable level of mRNA encoding the non-activating site-targeting receptor or ligand and a detectable level of the non-activating site targeting receptor or ligand protein.

In the context of the present invention, the terms “amount” and “level” are interchangeable.

### Reduced Expression Level of FAS

It will be appreciated that the “reduced expression level of FAS” of the genetically engineered Treg specifies a reduced level of expression of FAS relative to a non-genetically engineered Treg. A reduced level of expression of FAS may comprise a reduced level of FAS mRNA. In some embodiments, a reduced level of expression of FAS comprises a reduced level of FAS protein. Preferably, a reduced level of expression of FAS comprises a reduced level of FAS mRNA and FAS protein.

In some embodiments, the expression level of FAS is reduced by at least about 50%. In some embodiments, the expression level of FAS is reduced by at least about 60%, at least about 70%, at least about 80%, at least about 85%, at least about 90%, at least about 95% or at least about 99%. In some embodiments, the expression level of FAS is reduced by at least about 60%. In some embodiments, the expression level of FAS is reduced by at least about 80%. In some embodiments, the expression level of FAS is reduced by at least about 90%.

In some embodiments, the expression level of FAS is reduced by no more than about 99%, no more than about 95%, no more than about 90%, no more than about 85% or no more than about 80%. The expression level of FAS may be reduced by no more than about 85%, optionally no more than about 80%. In some embodiments, the expression level of FAS is reduced by of from about 50% to about 99%. Optionally, the expression level of FAS is reduced by of from about 50% to about 95%. Further optionally, the expression level of FAS is reduced by of from about 50% to about 90%.

The expression level of FAS may comprise a mean fluorescence intensity (MFI). Mean fluorescence intensity is a value calculated from flow cytometry which specifies the average fluorescence intensity for a protein per cell. For example, prior to genetic engineering, a Treg may comprise a FAS MFI of 10. If, after genetic engineering according to the invention, the Treg comprises a FAS MFI of 5, this will be understood to be a 50% reduction in the FAS expression level.

Prior to genetic engineering, a Treg may comprise a FAS MFI of at least about 3000, at least about 4000, at least about 5000, at least about 6000, at least about 7000, at least about 8000, at least about 9000, at least about 10000, at least about 11000 or at least about 12000. Prior to genetic engineering, a Treg may comprise a FAS MFI of no more than about 20000, no more than about 19000, no more than about 18000, no more than about 17000, no more than about 16000, no more than about 15000, no more than about 14000, no more than about 13000 or no more than about 12000.

Prior to genetic engineering, a Treg may comprise a FAS MFI of from about 3000 to about 12000.

The genetically engineered Treg of the invention may comprise a FAS MFI of no more than about 2000, no more than about 1500, no more than about 1000, no more than about 900, no more than about 800, no more than about 700, no more than about 600, no more than about 500, no more than about 400, no more than about 300, no more than about 200, no more than about 100 or no more than about 50. In some embodiments, the genetically engineered Treg may comprise a FAS MFI of from about 50 to about 2000, optionally of from about 200 to about 2000. Preferably, the genetically engineered Treg comprises a FAS MFI of from about 200 to about 1500, more preferably of from about 200 to about 1000. As the skilled person will appreciate, these MFI values are typically much lower than the FAS MFI values of a non-genetically engineered Treg and so represent a reduced expression level of FAS.

The expression level of FAS may be reduced by at least about 0.1 fold, at least about 0.2 fold, at least about 0.3 fold, at least about 0.4 fold, at least about 0.5 fold, at least about 0.6 fold, at least about 0.7 fold, at least about 0.8 fold, at least about 0.9 fold or at least about 0.95 fold.

The expression level of FAS may be reduced by no more than about 0.05 fold, optionally by no more than about 0.1 fold.

In some embodiments, the expression level of FAS is reduced by of from about 0.5 fold to about 0.05 fold. In some embodiments, the expression level of FAS is reduced by of from about 0.6 fold to about 0.05 fold. Optionally, the expression level of FAS is reduced by of from about 0.6 fold to about 0.1 fold.

The calculation of fold reduction is well known to the skilled person. For example, a reduction of at least about 0.1 fold will be understood to refer to the expression level of FAS being reduced by 10×, such that the expression level of FAS in the genetically engineered Treg is 10% of the expression level of the Treg prior to genetic engineering.

In some embodiments, the reduced expression level of FAS is an undetectable expression level of FAS. Prior to genetic engineering, the Treg may comprise a detectable expression level of FAS.

In some embodiments, the Treg is genetically engineered to express a molecule which reduces the expression level of FAS. The molecule may comprise or consist of a nucleotide sequence which reduces the expression level of FAS, a nuclease such as a transcription activator-like effector nuclease (TALEN) targeted to a target region of the endogenous FAS gene in the Treg cell, or a morpholino targeted to a target region of the endogenous FAS gene in the Treg cell.

By “endogenous FAS gene”, this will be understood to refer to the wild-type FAS gene native to the Treg cell. Thus, the endogenous FAS gene is present in the cell prior to genetic engineering; it is naturally occurring to the cell.

Preferably, the Treg is genetically engineered to express a nucleotide sequence which reduces the expression level of FAS. The nucleotide sequence which reduces the expression level of FAS may be complementary to a target region of the endogenous FAS gene in the Treg cell.

In the context of the present invention, the endogenous FAS gene may comprise endogenous FAS DNA. The endogenous FAS gene may comprise endogenous FAS mRNA. In embodiments where the nucleotide sequence which reduces the level of FAS is complementary to a target region of the endogenous FAS DNA, the nucleotide sequence is capable of binding to the target region of the FAS DNA and preventing or reducing transcription of the FAS gene. When the nucleotide sequence which reduces the expression level of FAS is complementary to a target region of the endogenous FAS mRNA, the nucleotide sequence is capable of binding to a target region of the FAS mRNA and preventing or reducing translation of the FAS gene.

The nucleotide sequence which reduces the expression level of FAS may comprise or consist of DNA and/or RNA. Preferably, the nucleotide sequence which reduces the expression level of FAS comprises or consists of RNA.

The nucleotide sequence may comprise or consist of short hairpin RNA (shRNA), an antisense oligonucleotide, double stranded RNA (dsRNA), or a CRISPR guide RNA.

Antisense oligonucleotides are single-stranded deoxyribonucleotides (DNA) which are complementary to a target region of an mRNA. Thus, in embodiments where the nucleotide sequence which reduces the expression level of FAS comprises or consists of an antisense oligonucleotide, the antisense oligonucleotide is complementary to a target region of endogenous FAS mRNA. Thus, when introduced into the cell, the antisense oligonucleotide forms an antisense oligonucleotide FAS mRNA duplex. The formation of this duplex induces RNAse H endonuclease activity which cleaves the duplex leading to a reduced level of translation of the FAS mRNA, in turn leading to a reduced expression level of FAS.

CRISPR (Class 2 Clustered Regularly Interspaced Short Palindromic Repeat) systems comprise a guide RNA (gRNA or sgRNA) and a CRISPR-associated endonuclease (Cas protein). The guide RNA comprises a scaffold sequence capable of binding to the CAS protein and a target-specific sequence complementary to the target DNA sequence to be modified. When introduced into the cell, the gRNA and the Cas protein form a complex which specifically binds to the target DNA sequence, enabling cleavage of the target DNA sequence by the Cas protein. This cleavage is then repaired by one of two general repair pathways which typically introduce nucleotide insertions, deletions or frameshift mutations which can lead to reduced expression of the target DNA sequence.

Thus, in some embodiments the nucleotide sequence comprises or consists of a CRISPR guide RNA. In the context of the present invention, the CRISPR guide RNA comprises a target-specific sequence complementary to a target region of the FAS gene in the Treg. In embodiments comprising a CRISPR guide RNA, the Treg may be genetically engineered to also express a CAS protein. The CAS protein may comprise a CAS9 protein.

In some embodiments the nucleotide sequence comprises or consists of dsRNA or shRNA. As the skilled person will appreciate, a short hairpin RNA (which may otherwise be referred to as a small hairpin RNA or hairpin vector) is an artificial single-stranded RNA sequence with complementary regions spaced by a short loop, causing the single-stranded RNA sequence to fold back upon itself to form a tight hairpin turn. shRNA or dsRNA can be used to silence target gene expression via RNA interference (RNAi). In RNAi, dsRNA or shRNA is bound and cleaved by the ribonuclease protein Dicer to produce double stranded fragments called siRNAs. The siRNAs are separated into single strands and integrated into a RISC complex, then base-pair and cleave the target mRNA, thereby reducing expression of the target gene.

Preferably, the nucleotide sequence which reduces the expression level of FAS comprises or consists of shRNA. The shRNA comprises an RNA sequence complementary to a target region of FAS mRNA. Once the shRNA is processed in the cell to form an siRNA, the siRNA comprises the RNA sequence complementary to the target region of FAS mRNA.

In some embodiments, the shRNA is encoded by the nucleotide sequence SEQ ID NO: 1 (GCGTATGACACATTGATTAAA), or a functional variant thereof. In some embodiments, the shRNA is encoded by the nucleotide sequence SEQ ID NO: 2 (GTGCAGATGTAAACCAAACTT), or a functional variant thereof.

As used herein, the term “variant” in the context of a nucleotide sequence encompasses a nucleotide sequence which is a naturally occurring polymorphic form of the basic sequence as well as synthetic variants, in which one or more nucleotides within the sequence are inserted, removed or replaced. In the context of the present invention, reference to a functional variant specifies that the molecule encoded by the variant substantially retains the functional activity of the molecule encoded by the basic sequence. “Substantially retains” will be understood to refer to a functional activity of at least about 80%, at least about 85%, at least about 90%, at least about 95%, at least about 97%, at least about 98%, at least about 99% or at least about 100% of the molecule encoded by the basic sequence. A functional variant of the present invention may have a functional activity equivalent or improved to the basic sequence. Functional variants also encompass truncated versions of the nucleotide sequence. Truncated versions of the nucleotide sequence are shortened versions of the basic nucleotide sequence which produces a biological effect in the encoded molecule which is equivalent to or improved relative to the molecule encoded by the basic sequence.

The functional variant may have at least about 60% sequence identity to the original sequence (for example, SEQ ID NO: 1 or SEQ ID NO:2). The functional variant may have at least about 70% sequence identity, at least about 75% sequence identity, at least about 80% sequence identity, at least about 90% sequence identity or at least about 95% sequence identity to the original sequence. In some embodiments the functional variant has at least about 90% sequence identity to the original sequence. In some embodiments, the functional variant has at least about 95% sequence identity to the original sequence. The functional variant may have of from about 75% sequence identity to about 95% sequence identity to the original sequence. In some embodiments the functional variant may have of from about 80% sequence identity to about 95% sequence identity to the original sequence.

In some embodiments, the nucleotide sequence which reduces the expression level of FAS comprises or consists of a microRNA (miRNA) comprising a shRNA.

As the skilled person will appreciate, a microRNA (miRNA) is a small (typically between 20 and 30 nucleotides in length) non-coding RNA which is capable of specifically hybridising to a target mRNA. The miRNA “specifically hybridises” to its target mRNA when it hybridises with preferential or high affinity to the target mRNA but does not substantially hybridise, does not hybridise, or hybridises with only low affinity to other polynucleotides, especially other non-target mRNAs. Typically, hybridisation of a miRNA to a target mRNA is due to substantially complementary base-pairing between the miRNA and the target mRNA. The hybridisation of the miRNA to the target RNA induces the degradation and translational repression of the target mRNA, thereby reducing translation of the target mRNA and expression of the protein which it encodes.

Since shRNA is artificial, it will be appreciated that the miRNA comprising a shRNA is also artificial. The miRNA comprising a shRNA may comprise a naturally occurring miRNA which has been genetically engineered to comprise a shRNA.

Typically, in nature, a primary miRNA, termed a pri-miRNA, is processed intracellularly to form a precursor miRNA (pre-miRNA). Within the cell, the pre-miRNA is further processed to form the final mature miRNA duplex. Pri-miRNA and pre-miRNA comprise a single-stranded RNA sequence with complementary regions spaced by a short loop, causing the single-stranded RNA sequence to fold back upon itself to form a stem-loop structure. Pri-miRNA and pre-miRNA may therefore comprise a 5′ stem sequence, a loop sequence and a 3′ stem sequence. The 5′ and 3′ stem sequences may be complementary to each other to form the double-stranded stem structure. The pri-miRNA may further comprise flanking sequences at the terminal 5 and 3′ ends of the sequence. Thus, the flanking sequences may be 5′ to the 5′ stem sequence and 3′ to the 3′ stem sequence.

Thus, in some embodiments the nucleotide sequence comprises a microRNA (miRNA), pre-miRNA and/or pri-miRNA comprising a shRNA.

Preferably, the nucleotide sequence which reduces the expression level of FAS comprises or consists of a pri-miRNA comprising a shRNA. Thus, when in the cell, the pri-miRNA is processed to form a pre-miRNA comprising the shRNA, which is then processed to form a miRNA comprising the shRNA or processed to form an miRNA and a shRNA.

In some embodiments, the nucleotide sequence which reduces the expression level of FAS comprises or consists of a pre-miRNA comprising a shRNA.

In some embodiments, the 5′ stem sequence of the pri-miRNA comprises the shRNA. In some embodiments, the 3′ stem sequence of the pri-miRNA comprises the shRNA.

Preferably, the loop sequence of the pri-miRNA comprises the shRNA. In such embodiments, the pri-miRNA is processed intracellularly to form a pre-miRNA wherein the loop sequence of the pre-miRNA comprises the shRNA. The pre-miRNA sequence is then processed intracellularly to cleave the loop structure from the stem sequences to form a final miRNA duplex and a separate shRNA nucleotide sequence. Without wishing to be bound by theory, the inventors believe that incorporation of the shRNA into the loop sequence of the pri/pre-miRNA avoids disturbance of the natural miRNA process, thereby avoiding overloading miRNA machinery in the cell. This may improve the viability of the cell and also improve efficiency of reduction of FAS expression in the cell.

In some embodiments, the 5′ stem sequence of the pre-miRNA comprises the shRNA. In some embodiments, the 3′ stem sequence of the pre-miRNA comprises the shRNA. Preferably, the loop sequence of the pre-miRNA comprises the shRNA.

Various naturally occurring miRNAs (and their precursor pri-miRNAs and/or pre-miRNAs) are known to the skilled person and are suitable as a “scaffold” which can be genetically engineered to incorporate an shRNA. Exemplary miRNAs, and their corresponding pre and pri-miRNAs include, but are not necessarily limited to those listed in Table 1.

TABLE 1
Exemplary miRNAs

| | Corresponding | Corresponding |
|---|---|---|
| miRNA | pre-miRNA | pri-miRNA |
| miR-30a | Pre- miR-30a | Pri- miR-30a |
| miR-30b | Pre- miR-30b | Pri- miR-30b |
| miR-30c-1 | Pre- miR-30c-1 | Pri- miR-30c-1 |
| miR-30c-2 | Pre- miR-30c-2 | Pri- miR-30c-2 |
| miR-30d | Pre- miR-30d | Pri- miR-30d |
| miR-30e | Pre- miR-30e | Pri- miR-30e |
| miR-451 | Pre- miR-451 | Pri- miR-451 |
| miR-328 | Pre- miR-328 | Pri- miR-328 |
| miR-223 | Pre- miR-223 | Pri- miR-223 |
| miR-1 | Pre- miR-1 | Pri- miR-1 |
| miR-133 | Pre- miR-133 | Pri- miR-133 |
| miR-208 | Pre- miR-208 | Pri- miR-208 |
| miR-21 | Pre- miR-21 | Pri- miR-21 |
| miR-17 | Pre- miR-17 | Pri- miR-17 |
| miR-92 | Pre- miR-92 | Pri- miR-92 |
| miR-34 | Pre- miR-34 | Pri- miR-34 |
| miR-122 | Pre- miR-122 | Pri- miR-122 |
| miR-375 | Pre- miR-375 | Pri- miR-375 |
| miR-103 | Pre- miR-103 | Pri- miR-103 |
| miR-107 | Pre- miR-107 | Pri- miR-107 |
| miR-106b | Pre- miR-106b | Pri- miR-106b |
| miR-93 | Pre- miR-93 | Pri- miR-93 |
| miR-25 | Pre- miR-25 | Pri- miR-25 |

In some embodiments, the nucleotide sequence which reduces the expression level of FAS comprises or consists of any of the miRNAs, pre-miRNAs and/or pri-miRNAs listed in Table 1 comprising a shRNA.

5 In some embodiments, the nucleotide sequence which reduces the expression level of FAS comprises or consists of a miR-30 miRNA comprising a shRNA.

MiR-30 miRNAs may include miR-30a, miR-30b, miR-30c-1, miR-30c-2, miR-30d and miR-30e.

In some embodiments the nucleotide sequence which reduces the expression level of FAS comprises or consists of a miR30a miRNA comprising a shRNA.

In some embodiments the nucleotide sequence which reduces the expression level of FAS comprises or consists of a miR30b miRNA comprising a shRNA.

In some embodiments the nucleotide sequence which reduces the expression level of FAS comprises or consists of a miR30c-1 miRNA comprising a shRNA.

In some embodiments the nucleotide sequence which reduces the expression level of FAS comprises or consists of a miR30c-2 miRNA comprising a shRNA.

In some embodiments the nucleotide sequence which reduces the expression level of FAS comprises or consists of a miR30d miRNA comprising a shRNA.

In some embodiments the nucleotide sequence which reduces the expression level of FAS comprises or consists of a miR30e miRNA comprising a shRNA.

Preferably, the nucleotide sequence which reduces the expression level of FAS comprises or consists of a pre-miR-30 pre-miRNA comprising a shRNA. Pre-miR-30 pre-miRNAs may include pre-miR-30a, pre-miR-30b, pre-miR-30c-1, pre-miR-30c-2, pre-miR-30d and pre-miR-30e.

In some embodiments the nucleotide sequence which reduces the expression level of FAS comprises or consists of a pre-miR30a pre-miRNA comprising a shRNA. An exemplary pre-miR30a pre-miRNA may comprise SEQ ID NO: 3 (GCGACUGUAAACAUCCUCGACUGGAAGCUGUGAAGCCACAGAUGGGCUUUCAGUCGGAUGUUU GCAGCUGC) or a functional variant thereof.

In some embodiments the nucleotide sequence which reduces the expression level of FAS comprises or consists of a pre-miR30b pre-miRNA comprising a shRNA. An exemplary pre-miR30b pre-miRNA may comprise SEQ ID NO: 4 (ACCAAGUUUCAGUUCAUGUAAACAUCCUACACUCAGCUGUAAUACAUGGAUUGGCUGGGAGGUG GAUGUUUACUUCAGCUGACUUGGA) or a functional variant thereof.

In some embodiments the nucleotide sequence which reduces the expression level of FAS comprises or consists of a pre-miR30c-1 pre-miRNA comprising a shRNA. An exemplary pre-miR30c-1 pre-miRNA may comprise SEQ ID NO: 5 (ACCAUGCUGUAGUGUGUGUAAACAUCCUACACUCUCAGCUGUGAGCUCAAGGUGGCUGGGAGA GGGUUGUUUACUCCUUCUGCCAUGGA) or a functional variant thereof.

In some embodiments the nucleotide sequence which reduces the expression level of FAS comprises or consists of a pre-miR30c-2 pre-miRNA comprising a shRNA. An exemplary pre-miR30c-2 pre-miRNA may comprise SEQ ID NO: 6 (AGAUACUGUAAACAUCCUACACUCUCAGCUGUGGAAAGUAAGAAAGCUGGGAGAAGGCUGUUUA CUCUUUCU) or a functional variant thereof.

In some embodiments the nucleotide sequence which reduces the expression level of FAS comprises or consists of a pre-miR30d pre-miRNA comprising a shRNA. An exemplary pre-miR30d pre-miRNA may comprise SEQ ID NO: 7 (GUUGUUGUAAACAUCCCCGACUGGAAGCUGUAAGACACAGCUAAGCUUUCAGUCAGAUGUUUGC UGCUAC) or a functional variant thereof.

In some embodiments the nucleotide sequence which reduces the expression level of FAS comprises or consists of a pre-miR30e pre-miRNA comprising a shRNA. An exemplary pre-miR30e pre-miRNA may comprise SEQ ID NO: 8 (GGGCAGUCUUUGCUACUGUAAACAUCCUUGACUGGAAGCUGUAAGGUGUUCAGAGGAGCUUUC AGUCGGAUGUUUACAGCGGCAGGCUGCCA) or a functional variant thereof.

The nucleotide sequence which reduces the expression level of FAS may comprise or consist of a pri-miR-30 pri-miRNA comprising a shRNA. Pri-miR-30 pri-miRNAs may include pri-miR-30a, pri-miR-30b, pri-miR-30c-1, pri-miR-30c-2, pri-miR-30d and pri-miR-30e.

In some embodiments the nucleotide sequence which reduces the expression level of FAS comprises or consists of a pri-miR30a pri-miRNA comprising a shRNA.

In some embodiments the nucleotide sequence which reduces the expression level of FAS comprises or consists of a pri-miR30b pri-miRNA comprising a shRNA.

In some embodiments the nucleotide sequence which reduces the expression level of FAS comprises or consists of a pri-miR30c-1 pri-miRNA comprising a shRNA.

In some embodiments the nucleotide sequence which reduces the expression level of FAS comprises or consists of a pri-miR30c-2 pri-miRNA comprising a shRNA.

In some embodiments the nucleotide sequence which reduces the expression level of FAS comprises or consists of a pri-miR30d pri-miRNA comprising a shRNA.

In some embodiments the nucleotide sequence which reduces the expression level of FAS comprises or consists of a pri-miR30e pri-miRNA comprising a shRNA.

The nucleotide sequence which reduces the expression level of FAS may comprise or consist of a pre-miR-30 pre-miRNA comprising a shRNA. Preferably, the nucleotide sequence which reduces the expression level of FAS comprises or consists of pre-miR-30 pre-miRNA comprising a shRNA in the loop sequence. In some embodiments, the nucleotide sequence which reduces the expression level of FAS comprises or consists of a pre miR-30 pre-miRNA comprising SEQ ID NO: 1, SEQ ID NO; 2 or a functional variant thereof. Most preferably, the nucleotide sequence which reduces the expression level of FAS comprises or consists of a pre miR-30 pre-miRNA comprising SEQ ID NO: 1, SEQ ID NO; 2 or a functional variant thereof in the loop sequence.

In some embodiments, the nucleotide sequence which reduces the expression level of FAS comprises or consists of a pre-miR-30a comprising a shRNA. In some embodiments, the nucleotide sequence which reduces the expression level of FAS comprises or consists of pre-miR-30a pre-miRNA comprising a shRNA in the loop sequence. In some embodiments, the nucleotide sequence which reduces the expression level of FAS comprises or consists of a pre miR-30a pre-miRNA comprising SEQ ID NO: 1, SEQ ID NO; 2 or a functional variant thereof. In some embodiments, the nucleotide sequence which reduces the expression level of FAS comprises or consists of a pre miR-30a pre-miRNA comprising SEQ ID NO: 1, SEQ ID NO; 2 or a functional variant thereof in the loop sequence.

Exemplary pre miR-30as comprising a shRNA include but are not necessarily limited to SEQ ID NOs 9 and 10. SEQ ID NOs 9 and 10 comprise pre-miR-30a pre-miRNA wherein FAS-targeting shRNA has been introduced into the loop sequence of the pre-miRNA. Thus, the nucleotide sequence which reduces the expression level of FAS may comprise or consist of SEQ ID NO: 9, SEQ ID NO: 10, or a functional variant thereof.

### Non-Activating Site-Targeting Receptor or Ligand

In accordance with the present invention, the Treg is genetically engineered to express a non-activating site-targeting receptor or ligand.

Preferably, the Treg is genetically engineered to express a non-activating site-targeting receptor. In some embodiments, the non-activating site-targeting receptor comprises or consists of a non-activating site-targeting antibody or functional variant thereof. In such embodiments, it will be appreciated that the antibody or functional variant thereof is a neutralising antibody or functional variant thereof. The antibody or functional variant thereof is optionally a blocking antibody or functional variant thereof.

The term “antibody” includes any molecule capable of specifically binding to an antigen. Thus, in embodiments where the non-activating site-targeting receptor or ligand comprises an antibody or functional variant thereof, the target comprises or consists of an antigen to which the antibody or functional variant thereof is capable of specifically binding.

The antibody may be a monoclonal antibody or a synthetic antibody, or other antibody mimetic, an aptamer, a protein scaffold or a major histocompatibility complex (MHC) protein or portion thereof. The antibody may be a small single chain antibody fragment (scFv); a full length antibody; an antibody fragment; an IgG; an scFv comprising an Fc region; an scFV-IGg; a diabody; a nanobody; an affibody; a single chain antibody lacking an Fc domain. In preferred embodiments the antibody is a small antibody such as an antibody fragment, for example and in particular an scFv. In some preferred embodiments the antibody is an scFV or other antibody that can be transcribed and/or translated from a single promoter and/or initiation signal compared to antibodies that may need to be transcribed and/or translated from two or more promoters and/or initiation signals.

As used herein, the term “variant” in the context of a protein (such as an antibody) encompasses a protein sequence which is a naturally occurring polymorphic form of the basic sequence as well as synthetic variants, in which one or more amino acids within the sequence are inserted, removed or replaced. In the context of the present invention, reference to a functional variant specifies that the molecule encoded by the variant substantially retains the functional activity of the molecule encoded by the basic sequence. “Substantially retains” will be understood to refer to a functional activity of at least about 80%, at least about 85%, at least about 90%, at least about 95%, at least about 97%, at least about 98%, at least about 99% or at least about 100% of the molecule encoded by the basic sequence. A functional variant of the present invention may have a functional activity equivalent or improved to the basic sequence. Functional variants also encompass truncated versions of the protein.

The functional variant may have at least about 60% sequence identity to the original amino acid sequence. The functional variant may have at least about 70% sequence identity, at least about 75% sequence identity, at least about 80% sequence identity, at least about 90% sequence identity or at least about 95% sequence identity to the original sequence. In some embodiments the functional variant has at least about 90% sequence identity to the original sequence. In some embodiments, the functional variant has at least about 95% sequence identity to the original sequence. The functional variant may have of from about 75% sequence identity to about 95% sequence identity to the original sequence. In some embodiments the functional variant may have of from about 80% sequence identity to about 95% sequence identity to the original sequence.

A functional variant of an antibody may comprise a fragment of an antibody or a genetically engineered version of one or more fragments of the antibody, provided that the fragment is still capable of specifically binding to the target antigen of the original antibody. For example, the functional variant may comprise or consist of a variable region (Fv), a complementarity determining region (CDR), a Fab, a single chain antibody (scFv), a heavy chain variable region (VH), a light chain variable region (VL) and/or a single-domain antibody (VHH).

“Complementarity determining region” or “CDR” with regard to an antibody or functional variant thereof refers to a highly variable loop in the variable region of the heavy chain or the light chain of an antibody. CDRs can interact with the antigen conformation and largely determine binding to the antigen (although some framework regions are known to be involved in binding). The heavy chain variable region and the light chain variable region each contain 3 CDRs. “Heavy chain variable region” or “VH” refers to the fragment of the heavy chain of an antibody that contains three CDRs interposed between flanking stretches known as framework regions, which are more highly conserved than the CDRs and form a scaffold to support the CDRs. “Light chain variable region” or “VL” refers to the fragment of the light chain of an antibody that contains three CDRs interposed between framework regions.

“Fv” refers to the smallest fragment of an antibody to bear the complete antigen binding site. An Fv fragment consists of the variable region of a single light chain bound to the variable region of a single heavy chain. “Single-chain Fv antibody” or “scFv” refers to an engineered antibody consisting of a light chain variable region and a heavy chain variable region connected to one another directly or via a peptide linker sequence.

Thus, in some embodiments the non-activating site-targeting receptor comprises a non-activating site-targeting antibody, Fv, Fab or scFv. In some embodiments the non-activating site-targeting receptor comprises a non-activating site targeting antibody or scFv. In some embodiments the non-activating site-targeting receptor comprises a non-activating site-targeting scFV.

As described herein, the site to which the non-activating receptor or ligand is targeted may comprise a specific anatomical location. Alternatively, the site may comprise a particular cell population. Preferably, the non-activating site-targeting receptor comprises a non-activating anatomical location-targeting antibody, Fv, Fab or scFV. In such embodiments, the antibody, Fv, Fab or scFV is capable of specifically binding to an antigen expressed and specific to the particular anatomical location. This ensures that, once introduced into a subject, the Treg is directed to the anatomical location.

In embodiments where the site comprises an anatomical location, the site may comprise one or more of bone marrow, joints, cartilage, bone, tumour microenvironment (TME), central nervous system (CNS), peripheral nervous system, eyes, thyroid, salivary glands, lungs, liver, bowel, bladder, colon, rectum, large intestine, small intestine, skin, blood and stomach. As used herein, the term CNS refers to the central nervous system, which comprises the brain and spinal cord.

In some embodiments, the site comprises one or more of bone marrow, tumour microenvironment (TME), central nervous system (CNS), lungs, liver, bowel, bladder, large intestine, skin, small intestine and stomach. The site may be selected from bone marrow, TME, central nervous system, large intestine and small intestine.

In some embodiments, the anatomical location is an inflamed anatomical location. By “inflamed anatomical location”, this will be understood to refer to an anatomical site in the subject where inflammation is occurring or has occurred. Such inflamed anatomical locations may be present in autoimmune disease, infection or cancer.

Preferably, the site comprises or consists of bone marrow. Thus, in some embodiments, the non-activating site-targeting receptor or ligand comprises a non-activating bone-marrow targeting receptor or ligand. Preferably, the non-activating site-targeting receptor or ligand comprises a non-activating bone-marrow targeting receptor. More preferably, the non-activating site-targeting receptor or ligand comprises a non-activating bone-marrow targeting antibody or functional variant thereof.

The non-activating bone-marrow targeting antibody may comprise a non-activating anti-CD271, anti-CD106, anti-STRO-1, anti-CD146, anti-CD90, anti-CD105, anti-frizzle-9, anti-SSEA-4, anti-CD51, anti-CD140a, anti-SUSD2, anti-MSCA-1, anti-CD230, anti-LEPR or anti-CD34 antibody. Most preferably, the non-activating site-targeting antibody or functional variant thereof is a non-activating anti-CD34 antibody or functional variant thereof.

As the skilled person will appreciate, CD34 is a transmembrane phosphoglycoprotein which is naturally expressed on hematopoietic stem cells, and so is localised to the bone marrow. As such, genetic engineering of the Treg to express a non-activating anti-CD34 antibody or functional variant thereof may advantageously localise the Tregs to the anatomical location of bone marrow, once introduced into a subject.

In some embodiments, the non-activating site-targeting receptor is a functional variant of an anti-CD34 antibody. The functional variant may comprise or consist of a non-activating anti-CD34 scFV.

Various anti-CD34 antibodies are known and commercially available. Exemplary anti-CD34 antibodies include but are not necessarily limited to the QBEND/10 anti-CD34 antibody and the EP373Y anti-CD34 antibody. In some embodiments the non-activating site-targeting antibody comprises the QBEND/10 anti-CD34 antibody. QBEND/10 is a mouse monoclonal antibody capable of specifically binding to the class II epitope of CD34. In particular, QBEND/10 is a neutralising anti-CD34 antibody. A known downstream effect of intracellular signalling activity from CD34 is tube formation in human umbilical vein endothelial cells (HUVECs). This can be measured using an in vitro HUVEC tube formation assay, which is known in the art. Hence, QBEND/10 is capable, upon specifically binding CD34 on HUVECs, of reducing tube formation in the HUVECs.

In some embodiments the non-activating site-targeting receptor or functional variant thereof comprises a heavy chain variable region comprising or consisting of SEQ ID NO: 11 or a functional variant thereof. SEQ ID NO 11 is a heavy chain variable region of the QBEND/10 anti-CD34 antibody. The functional variant may have at least about 80% sequence identity to SEQ ID NO: 11. In some embodiments, the functional variant has at least about 90%, optionally at least about 95% or 99% sequence identity to SEQ ID NO: 11.

In some embodiments the non-activating site-targeting receptor or functional variant thereof comprises a light chain variable region comprising or consisting of SEQ ID NO: 12 or a functional variant thereof. SEQ ID NO: 12 is a light chain variable region of the QBEND/10 anti-CD34 antibody. The functional variant may have at least about 80% sequence identity to SEQ ID NO: 12. In some embodiments, the functional variant has at least about 90%, optionally at least about 95% or 99% sequence identity to SEQ ID NO: 12.

In some embodiments the non-activating site-targeting receptor or functional variant thereof comprises SEQ ID NOs 11 and 12 or functional variants thereof. In some embodiments, the non-activating site-targeting receptor or functional variant thereof comprises, from 5′ to 3′ SEQ ID NO 11 and SEQ ID NO: 12. Alternatively, the non-activating site-targeting receptor or functional variant thereof may comprise, from 5′ to 3′ SEQ ID NO 12 and SEQ ID NO: 11. In embodiments comprising SEQ ID NOs 11 and 12 or functional variants thereof, SEQ ID NOs 11 and 12 or the functional variants thereof may be linked by a linker sequence. The linker sequence may comprise SEQ ID NO: 13. The linker sequence may be encoded by SEQ ID NO: 31, 32, 33 or 34.

In some embodiments, the non-activating site-targeting receptor comprises a functional variant of the QBEND/10 anti-CD34 antibody. The functional variant of the QBEND/10 aCD34 antibody may comprise or consist of a QBEND/10 anti-CD34 scFv.

The QBEND/10 αCD34 scFv may comprise or consist of SEQ ID NO: 14, SEQ ID NO: 15 or a functional variant thereof. In some embodiments, the QBEND/10 anti-CD34 scFv is encoded by a nucleotide sequence comprising or consisting of SEQ ID NO: 16, SEQ ID NO: 17 or a functional variant thereof.

The non-activating site-targeting receptor or ligand may be murine, human or humanised. Preferably, the non-activating site-targeting receptor or ligand is human or humanised.

In some embodiments, the non-activating site-targeting receptor or ligand comprises a humanised antibody or functional variant thereof. In some embodiments, the non-activating site-targeting receptor or ligand comprises a humanised anti-CD34 antibody or functional variant thereof. In some embodiments, the non-activating site-targeting receptor or ligand comprises a humanised QBEND/10 anti-CD34 antibody or functional variant thereof.

In some embodiments, the non-activating site-targeting receptor or ligand comprises an extracellular domain and a transmembrane domain. In some embodiments, the non-activating site-targeting receptor or ligand further comprises a hinge domain. The transmembrane domain may comprise a CD28 transmembrane domain. Optionally, the hinge domain comprises a CD28 hinge domain. The CD28 hinge domain may comprise or consist of SEQ ID NO: 18.

In some embodiments, the non-activating site-targeting receptor or ligand comprises a leader sequence. Various exemplary leader sequences will be known to those skilled in the art. In some embodiments, the leader sequence comprises or consists of a CD8a leader sequence. The CD8a leader sequence may comprise or consist of SEQ ID NO: 19.

Preferably, the non-activating site-targeting receptor or ligand does not comprise an intracellular domain.

In some embodiments, the non-activating site-targeting receptor or ligand further comprises a marker or tag amino acid sequence. Various markers are known and commercially available to the skilled person. By including a marker, the receptor or ligand can advantageously be readily detected upon expression in the cell. Exemplary markers may include, but not necessarily be limited to, a myc tag, luciferase or GFP. In some embodiments, the non-activating site-targeting receptor or ligand comprises a myc tag.

The non-activating site-targeting receptor or ligand may comprise a linker sequence. Numerous linker sequences are known and available to those skilled in the art. For example, the non-activating site-targeting receptor or ligand may comprise a G4S linker sequence. The linker sequence is preferably encoded by SEQ ID NO: 31, 32, 33 or 34.

In some embodiments, the non-activating site-targeting receptor or ligand is a non-activating site-targeting receptor comprising, from 5′ to 3′: a leader sequence, an extracellular sequence, a hinge domain, a myc tag and a transmembrane domain. In some embodiments, the non-activating site-targeting receptor or ligand is a non-activating site-targeting scFv comprising, from 5′ to 3′: a leader sequence, an extracellular heavy variable chain, an extracellular light variable chain, a hinge domain, a myc tag and a transmembrane domain.

In some embodiments, the non-activating site-targeting receptor or ligand is a non-activating site-targeting scFv comprising, from 5′ to 3′: a leader sequence, an extracellular light variable chain, an extracellular heavy variable chain, a hinge domain, a myc tag and a transmembrane domain.

The non-activating site-targeting receptor or ligand may be a non-activating site-targeting scFv comprising, from 5′ to 3′: a CD8a leader sequence, an extracellular light variable chain, an extracellular heavy variable chain, a CD28 hinge domain, a myc tag and a CD28 transmembrane domain. Alternatively, the non-activating site-targeting receptor or ligand may be a non-activating site-targeting scFv comprising, from 5′ to 3′: a CD8a leader sequence, an extracellular heavy variable chain, an extracellular light variable chain, a CD28 hinge domain, a myc tag and a CD28 transmembrane domain. The heavy and/or light chains may comprise or consist of QBEND10 heavy and/or light variable chains, for example SEQ ID NOs 11 and/or 12. The heavy and light chains may be linked by a linker sequence, for example a G4S linker sequence. In some embodiments the linker sequence comprises at least three G4S linker sequences. The linker sequence is preferably encoded by SEQ ID NO: 31, 32, 33 or 34.

### Treg Cell

In some embodiments, the Treg is genetically engineered to further express a chemokine receptor or a functional variant thereof.

As the skilled person will appreciate, expression of a chemokine receptor can facilitate chemotaxis, cell adhesion and mediator release by the Treg. Chemotaxis, in particular, may be beneficial to further target migration of the Treg to a specific target site. The Treg may be genetically engineered to express any suitable chemokine receptor or functional variant thereof.

Chemokine receptors may comprise conventional chemokine receptors (cCKRs) and atypical chemokine receptors (ACKRs). In some embodiments, the chemokine receptor comprises a conventional chemokine receptor or a functional variant thereof.

Conventional chemokine receptors may comprise CCL receptors, XCL receptors, CXCL receptors and CX3CL receptors. Exemplary CCL receptors include, but are not necessarily limited to CCR1, CCR2, CCR2A, CCR2(B), CCR3, CCR4, CCR5, CCR6, CCR7, CCR8, CCR9A, CCR9(B) and CCR10. Exemplary CXCL receptors include, but are not necessarily limited to CXCR1, CXCR2, CXCR3 (a), CXCR3-alt, CXCR3B, CXCR4, CXCR5, CXCR6 and CXCR8. An exemplary XCL receptor includes XCR1. An exemplary CX3CL receptor is CX3CR1.

In some embodiments, the Treg is genetically engineered to further express a CXCL chemokine receptor or a functional variant thereof. Preferably, the Treg is genetically engineered to further express a CXCL chemokine receptor selected from CXCR1, CXCR2, CXCR3 (a), CXCR4, CXCR5, CXCR6 and CXCR8 or a functional variant thereof.

In some embodiments, the chemokine receptor comprises or consists of CXCR4 or a functional variant thereof. Expression of CXCR4 may facilitate chemotaxis of the cell to hematopoietic stem cells, which advantageously may further facilitate homing of the Treg to the bone marrow. The CXCR4 is preferably encoded by SEQ ID NO: 35.

In some embodiments, the Treg is genetically engineered to further express a puromycin-N-acetyltransferase (PAC). This may otherwise be referred to as a puromycin resistance gene. Expression of PAC confers puromycin resistance to the genetically engineered Tregs, meaning that when cultured in puromycin-comprising media, only Tregs which have been successfully genetically engineered and are expressing PAC will remain live.

The Treg may comprise or consist of a primary cell. By “primary cell” this will be understood to refer to a cell that has been obtained from a subject. Primary cells are not immortalised cells from a cell line. Optionally, the Treg is a primary human Treg.

The primary Treg may be autologous. Alternatively, the primary Treg may be allogeneic.

As the skilled person will appreciate, autologous cells are cells from the same subject, i.e., cells which have been obtained from a subject which will be administered back to the same subject. Allogeneic cells are cells obtained from a different subject to the subject to which the cells will be administered. The different subjects are typically from the same species. Allogenic cells are thus genetically different to the subject to which they are administered.

Alternatively, the Treg may comprise or consist of an immortalised Treg from a cell line.

The skilled person will be well aware of markers to identify a Treg cell. For example, the Treg may be FOXP3⁺. By “FOXP3⁺”, this will be understood to mean that the cell comprises a detectable expression level of FOXP3, such that the cell is considered “positive” for FOXP3 when analysed, for example, by flow cytometry. In some embodiments, the Treg is IL-10⁺. In some embodiments, the Treg is CD25⁺. In some embodiments, the Treg is CD127 lo or negative. As the skilled person will appreciate, CD127 is downregulated on Treg cells. Thus, reference to “CD127 lo” or “CD127 negative” specifies that the cell comprises an undetectable or “low” expression level of CD127. In the context of the present invention, a “CD127 lo” cell may be identified relative to a CD4⁺ T cell. CD4⁺ T cells express high levels of CD127. Thus, when flow cytometry is used for detection, the expression level of CD127 may be identified in a population of CD4⁺ T cells, then a threshold set below this expression level. When the expression level of CD127 is then assessed in Tregs, an expression level below the threshold may be classified as CD127 lo. In some embodiments, the Treg is CTLA-4⁺. In some embodiments, the Treg is GITR, Nrp1 and/or Helios⁺.

A Treg may also be defined by its functionality. As the skilled person will appreciate, Tregs are suppressive, anti-inflammatory T cells. In some embodiments, the genetically engineered Treg has increased suppressive activity (compared to a Treg prior to or without genetic engineering). Suppressive activity may be against, for example, effector CD4⁺ or CD8⁺ T cells. Suppressive activity may comprise suppression of proliferation of the effector T cells and/or suppression of secretion of inflammatory cytokines such as IFN-γ, IL-17, IL-2 and/or GM-CSF.

Suppressive activity may be increased by at least about 10%, at least about 20%, at least about 30%, at least about 40%, at least about 50%, at least about 60%, at least about 70%, at least about 80%, at least about 90%, at least about 95% or at least about 99%. In some embodiments, the suppressive activity of the genetically engineered Treg is increased by at least about 50%. The suppressive activity of the genetically engineered Treg may be increased by at least about 70%, optionally at least about 80%, or optionally at least about 90%.

The suppressive activity of a Treg can be determined by in vitro co-incubation of the Treg with PBMCs, CD4⁺ effector T cells or CD8⁺ T cells. Suppressive activity may be measured as a reduction in proliferation of the co-incubated PBMCs, CD4⁺ effector T cells or CD8⁺ T cells compared to PBMCs, CD4⁺ effector T cells or CD8⁺ T cells not incubated with the Tregs. Proliferation can be measured using various assays, including a thymidine assay. Other methods will be known to the skilled person. Alternatively, or in addition, suppressive activity may be measured as a reduction in the secretion of inflammatory cytokines from the co-incubated PBMCs, CD4⁺ effector T cells or CD8⁺ T cells compared to PBMCs, CD4⁺ effector T cells or CD8⁺ T cells not incubated with the Tregs. Cytokine secretion may be measured by ELISA, methods and kits for which are well known to the skilled person.

In some embodiments, the viability of the genetically engineered Treg is improved relative to a non-genetically engineered Treg or a Treg prior to genetic engineering. Viability may comprise the time for which the Treg is live. For example, the genetically engineered Treg may be viable for at least about two weeks, at least about three weeks or at least about four weeks. In some embodiments, the genetically engineered Treg is viable for at least about three weeks. The genetically engineered Treg may be viable for no more than about 12 weeks, no more than about 11 weeks or no more than about 10 weeks. Advantageously, the Tregs of the present invention are viable for longer than non-modified Tregs. This may improve therapeutic efficacy since the cells can remain functional and active for longer.

In some embodiments, the Treg is genetically engineered to express:
a) a non-activating site-targeting antibody or functional variant thereof; and
b) a nucleotide sequence comprising a shRNA which reduces the expression level of FAS.

In some embodiments, the Treg is genetically engineered to express:
a) a non-activating site-targeting antibody or functional variant thereof; and
b) a nucleotide sequence comprising a pri-miRNA or pre-miRNA comprising a shRNA which reduces the expression level of FAS.

In some embodiments, the Treg is genetically engineered to express:
a) a non-activating site-targeting anti-CD34 antibody or functional variant thereof; and
b) a nucleotide sequence comprising a pri-miRNA or pre-miRNA comprising a shRNA which reduces the expression level of FAS.

In some embodiments, the Treg is genetically engineered to express:
a) a non-activating site-targeting anti-CD34 scFv; and
b) a nucleotide sequence comprising a pri-miR30 or pre-miR30 comprising a shRNA which reduces the expression level of FAS.

In some embodiments, the Treg is genetically engineered to express:
a) a non-activating site-targeting anti-CD34 scFv; and
b) a nucleotide sequence comprising a shRNA encoded by SEQ ID NO: 1, SEQ ID NO: 2 or a functional variant thereof, which reduces the expression level of FAS.

In some embodiments, the genetically engineered Treg comprises a population of Tregs genetically engineered to express:
a) a non-activating site-targeting receptor or ligand; and
b) a reduced expression level of FAS.

Any number of cells may be present in the population. The population preferably comprises at least about 1×10⁵ Tregs. The population more preferably comprises at least about 5×10⁵, at least about 1×10⁶, at least about 1.5×10⁶, at least about 2×10⁶, at least about 5×10⁶, or at least about 1×10⁷ Tregs. In some embodiments, the population comprises no more than about 1×10⁹, no more than about 5×10⁸, no more than about 1×10⁸ or no more than about 5×10⁷ Tregs. In some embodiments, the population comprises of from about 1×10⁵ Tregs to about 1×10⁹ Tregs. In some embodiments, the population comprises of from about 1×10⁵ Tregs to about 1×10⁸ Tregs.

In embodiments comprising a population of Tregs, a reduced expression level of FAS may comprise a reduced percentage of cells expressing a detectable level of FAS. The reduced percentage of cells would be relative to the population of cells prior to genetic engineering.

In some embodiments, the proportion of Tregs expressing FAS is reduced by at least about 50%. In some embodiments, the proportion of Tregs expressing FAS is reduced by at least about 60%, at least about 70%, at least about 80%, at least about 85%, at least about 90%, at least about 95% or at least about 99%. In some embodiments the proportion of Tregs expressing FAS is reduced by at least about 60%. In some embodiments, the proportion of Tregs expressing FAS is reduced by at least about 80%.

In some embodiments, the proportion of Tregs expressing FAS is reduced by no more than about 99%, no more than about 95%, no more than about 90%, no more than about 85% or no more than about 80%. The proportion of Tregs expressing FAS may be reduced by no more than about 85%, optionally no more than about 80%. In some embodiments, the proportion of Tregs expressing FAS is reduced by of from about 50% to about 99%. Optionally, the proportion of Tregs expressing FAS is reduced by of from about 50% to about 95%. Further optionally, the proportion of Tregs expressing FAS is reduced by of from about 50% to about 90%.

Various methods are available to determine the proportion of Tregs expressing a detectable level of FAS. For example, flow cytometry may be used to determine the proportion of FAS⁺ cells. Other methods will be known and available to the skilled person.

The population of Tregs may comprise a population of primary Tregs. The population of primary Tregs may comprise a mixture of autologous and allogenic Tregs.

The present invention also provides a regulatory T-cell (Treg) genetically engineered to express a non-activating site-targeting receptor or ligand. The non-activating site-targeting receptor or ligand may be as defined above.

Also provided is a regulatory T-cell (Treg) genetically engineered to express a reduced expression level of FAS. The reduced expression level of FAS may be as defined above.

### Nucleotide Sequences

In some embodiments, the Treg comprises a nucleotide sequence encoding the non-activating site-targeting receptor or ligand.

The invention also provides a polynucleotide sequence comprising a nucleotide sequence which reduces the expression level of FAS as defined above. Also provided by the invention is a polynucleotide sequence comprising a nucleotide sequence encoding a non-activating site-targeting receptor or ligand as defined above. Any embodiments defined for the Treg cell above, especially the nucleotide sequence which reduces the expression level of FAS and the non-activating site-targeting receptor or ligand apply equally to the polynucleotide sequence.

It will be appreciated that the polynucleotide sequence(s) is/are recombinant. Preferably, the polynucleotide sequence(s) is/are isolated.

The invention also provides a or a plurality of polynucleotide sequence(s) comprising:
a) a nucleotide sequence which reduces the expression level of FAS; and
b) a nucleotide sequence encoding a non-activating site-targeting receptor or ligand.

Optionally, the polynucleotide or the plurality of polynucleotide sequence(s) further comprises a nucleotide sequence encoding a chemokine receptor or a functional variant thereof.

In some embodiments, one polynucleotide sequence comprises a nucleotide sequence which reduces the expression level of FAS and a nucleotide sequence encoding a non-activating site-targeting receptor or ligand. Optionally, the one polynucleotide sequence further comprises a nucleotide sequence encoding a chemokine receptor or a functional variant thereof.

In some embodiments the one polynucleotide sequence comprises, from 5′ to 3′, the nucleotide sequence encoding a non-activating site-targeting receptor or ligand and the nucleotide sequence which reduces the expression level of FAS. The one polynucleotide may further comprise a nucleotide sequence encoding a ribosomal skipping sequence between the nucleotide sequence encoding a non-activating site-targeting receptor or ligand and the nucleotide sequence which reduces the expression level of FAS.

The one polynucleotide sequence may further comprise a nucleotide sequence encoding a reporter gene. Suitable reporter genes include, but are not necessarily limited to luciferase, myc, HNIS, hNET and HSVtK. In some embodiments the one polynucleotide sequence further comprises a nucleotide sequence encoding luciferase and/or a nucleotide sequence encoding myc.

Alternatively, a plurality of polynucleotide sequences may comprise a nucleotide sequence which reduces the expression level of FAS and a nucleotide sequence encoding a non-activating site-targeting receptor or ligand. For example, the polynucleotide sequences may comprise a first polynucleotide sequence comprising a nucleotide sequence which reduces the expression level of FAS and a second polynucleotide sequence comprising a nucleotide sequence encoding a non-activating site-targeting receptor or ligand.

In some embodiments, the first polynucleotide sequence further comprises a nucleotide sequence encoding a chemokine receptor or a functional variant thereof. In some embodiments, the second polynucleotide sequence further comprises a nucleotide sequence encoding a chemokine receptor or a functional variant thereof. Alternatively, the plurality of polynucleotide sequences may further comprise a third polynucleotide sequence comprising a nucleotide sequence encoding a chemokine receptor or a functional variant thereof.

In some embodiments, the polynucleotide sequence(s) further comprises a nucleotide sequence encoding PAC.

A polynucleotide, such as a nucleic acid, is a polymer comprising two or more nucleotides. The nucleotides can be naturally occurring or artificial.

A nucleotide typically contains a nucleobase, a sugar and at least one linking group, such as a phosphate, 2′O-methyl, 2′ methoxy-ethyl, phosphoramidate, methylphosphonate or phosphorothioate group. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The sugar and the nucleobase together form a nucleoside. Preferred nucleosides include, but are not limited to, adenosine, guanosine, 5-methyluridine, uridine, cytidine, deoxyadenosine, deoxyguanosine, thymidine, deoxyuridine and deoxycytidine. The nucleosides may be adenosine, guanosine, uridine and cytidine.

The nucleotides are typically ribonucleotides or deoxyribonucleotides. The nucleotides may be deoxyribonucleotides. The nucleotides typically contain a monophosphate, diphosphate or triphosphate. Phosphates may be attached on the 5′ or 3′ side of a nucleotide.

Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate, cyclic adenosine monophosphate (CAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate. The nucleotides may be selected from AMP, UMP, GMP, CMP, dAMP, dTMP, dGMP or dCMP. In some embodiments, the nucleotides are selected from dAMP, dTMP, dGMP or dCMP.

The nucleotides may contain additional modifications. In particular, suitable modified nucleotides include, but are not limited to, 2′amino pyrimidines (such as 2′-amino cytidine and 2′-amino uridine), 2′-hydroxyl purines (such as, 2′-fluoro pyrimidines (such as 2′-fluorocytidine and 2′fluoro uridine), hydroxyl pyrimidines (such as 5′-α-P-borano uridine), 2′-O-methyl nucleotides (such as 2′-O-methyl adenosine, 2′-O-methyl guanosine, 2′-O-methyl cytidine and 2′-O-methyl uridine), 4′-thio pyrimidines (such as 4′-thio uridine and 4′-thio cytidine) and nucleotides have modifications of the nucleobase (such as 5-pentynyl-2′-deoxy uridine, 5-(3-aminopropyl)-uridine and 1,6-diaminohexyl-N-5-carbamoylmethyl uridine).

One or more nucleotides in the polynucleotide(s) may be modified, for instance with a label or a tag. The label may be any suitable label which allows the nucleotides to be detected. Suitable labels include, but are not limited to, fluorescent molecules, radioisotopes, e.g., ¹²⁵I, ³⁵S, enzymes, antibodies, antigens, other polynucleotides and ligands such as biotin.

The nucleotides in the polynucleotide(s) may be attached to each other in any manner. The nucleotides may be linked by phosphate, 2′O-methyl, 2′ methoxy-ethyl, phosphoramidate, methylphosphonate or phosphorothioate linkages. The nucleotides are typically attached by their sugar and phosphate groups. The nucleotides may be connected via their nucleobases as in pyrimidine dimers.

The polynucleotide(s) may comprise a deoxyribonucleic acid (DNA) or a ribonucleic acid (RNA). The polynucleotide(s) may be any synthetic nucleic acid known in the art, such as peptide nucleic acid (PNA), glycerol nucleic acid (GNA), threose nucleic acid (TNA), locked nucleic acid (LNA), morpholino nucleic acid or other synthetic polymers with nucleotide side chains.

Unless otherwise indicated, a particular polynucleotide sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with a different base, mixed-base and/or deoxyinosine residues (Batzer et al., 1991, Nucleic Acid Res. 19:5081; Ohtsuka et al., 1985, J. Biol. Chem. 260:2605-2608; and Rossolini et al., 1994, Mol. Cell. Probes 8:91-98).

Substitutions may be used for the practices of codon optimisation and codon wobble, both of which are known to those skilled in the art. Thus, it will be appreciated that codon-optimised and codon-wobbled isolated polynucleotide(s) are also envisaged. In an embodiment, the polynucleotide(s) is/are codon-optimised for human expression.

The polynucleotide(s) can be produced by de novo solid-phase DNA synthesis or by PCR mutagenesis of an existing sequence. Direct chemical synthesis of polynucleotides can be accomplished by methods known in the art, such as the phosphotriester method of Narang et al., 1979, Meth. Enzymol. 68:90; the phosphodiester method of Brown et al., 1979, Meth. Enzymol. 68:109; the diethylphosphoramidite method of Beaucage et al., 1981, Tetra. Lett., 22:1859; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif, 1990; Mattila et al., 1991, Nucleic Acids Res. 19:967; and Eckert et al., 1991, PCR Methods and Applications 1:17.

### Vector(s)

In some embodiments, the Treg comprises a vector encoding the nucleotide sequence which reduces the expression level of FAS. In some embodiments, the Treg comprises a vector encoding the nucleotide sequence encoding the non-activating site-targeting receptor or ligand.

In some embodiments, the Treg comprises one vector encoding the nucleotide sequence which reduces the expression level of FAS and the nucleotide sequence encoding the non-activating site-targeting receptor or ligand. Genetic engineering of the Treg using only one vector minimises modification time and stress upon the cell in vitro, which may improve the cell viability and efficacy.

Alternatively, the Treg may comprise a first vector encoding the nucleotide sequence which reduces the expression level of FAS and a second vector comprising a nucleotide sequence encoding the non-activating site-targeting receptor or ligand.

In embodiments where the Treg is genetically engineered to further express a chemokine receptor or a functional variant thereof, the Treg may comprise a nucleotide sequence encoding the chemokine receptor or functional variant thereof. In some embodiments, the Treg comprises a vector encoding the nucleotide sequence encoding the chemokine receptor or functional variant thereof.

In some embodiments, the Treg comprises a vector encoding the nucleotide sequence which reduces the expression level of FAS and a nucleotide sequence encoding the chemokine receptor or functional variant thereof. In some embodiments, the Treg comprises a vector encoding the nucleotide sequence encoding the non-activating site-targeting receptor or ligand and a nucleotide sequence encoding the chemokine receptor or functional variant thereof.

In some embodiments the Treg comprises a first vector encoding the nucleotide sequence which reduces the expression level of FAS and a nucleotide sequence encoding the chemokine receptor or functional variant thereof and a second vector encoding the non-activating site-targeting receptor or ligand. Alternatively, the Treg may comprise a first vector encoding the nucleotide sequence encoding the non-activating site-targeting receptor or ligand and a nucleotide sequence encoding the chemokine receptor or functional variant thereof and a second vector encoding the nucleotide sequence which reduces the expression level of FAS. In some embodiments, the Treg comprises one vector encoding the nucleotide sequence which reduces the expression level of FAS, a nucleotide sequence encoding the chemokine receptor or functional variant thereof and a nucleotide sequence encoding the non-activating site-targeting receptor or ligand.

The vector(s) may comprise any of the polynucleotide sequence(s) disclosed above. For example, the vector(s) may comprise a nucleotide sequence encoding PAC.

Also provided by the invention is a vector comprising the polynucleotide sequence(s)/nucleotide sequences disclosed above. Any embodiments defined for the polynucleotide sequence(s) above apply equally to the vector. Likewise, as described above, the vector may comprise one vector comprising the polynucleotide sequence(s). For example, the vector may comprise one vector comprising a nucleotide sequence which reduces the expression level of FAS and the nucleotide sequence encoding the non-activating site-targeting receptor or ligand. The one vector may further comprise a nucleotide sequence encoding the chemokine receptor or functional variant thereof.

Alternatively, the vector may comprise a plurality of vectors comprising the polynucleotide sequence(s), as defined above in relation to a first, second, and optional third vector.

In some embodiments, the vector(s) is an expression vector. Various expression vectors can be employed to express the nucleotide sequence encoding the non-activating site-targeting receptor or ligand, nucleotide sequence which reduces the expression level of FAS and/or nucleotide sequence encoding the chemokine receptor or functional variant thereof.

Both viral-based and non-viral expression vectors can be used to introduce the above-noted nucleotide sequences into the Treg. Non-viral vectors and systems include plasmids, episomal vectors, typically with an expression cassette for expressing a protein or RNA, and human artificial chromosomes (see, e.g., Harrington et al., 1997, Nat Genet. 15:345). For example, non-viral vectors useful for expression in mammalian (e.g., human) cells include pThioHis A, B and C, pcDNA3.1/His, pEBVHis A, B and C, (Invitrogen, San Diego, Calif.), MPS V vectors, and numerous other vectors known in the art for expressing other proteins and/or nucleotide sequences. Useful viral vectors include vectors based on retroviruses, adenoviruses, adeno-associated viruses, herpes viruses, vectors based on SV40, papilloma virus, HBP Epstein Barr virus, vaccinia virus vectors and Semliki Forest virus (SFV). See, Brent et al., supra; Smith, 1995, Annu. Rev. Microbiol. 49:807; and Rosenfeld et al., 1992, Cell 68:143.

Preferably, the vector(s) is a retroviral, lentiviral, adenoviral or adeno-associated viral vectors Examples of such vectors include the SFG retroviral expression vector (see Riviere et al., 1995, Proc. Natl. Acad. Sci. (USA) 92:6733-6737).

In some embodiments, the vector is a retroviral or lentiviral vector. In some embodiments, the vector is a retroviral vector. Optionally, the vector is an SFG retroviral vector. In some embodiments the vector is a lentiviral vector. Lentiviral vectors include self-inactivating lentiviral vectors (so-called SIN vectors).

Expression vectors for mammalian host cells can include expression control sequences, such as an origin of replication, a promoter, and an enhancer (see, e.g., Queen, et al., 1986, Immunol. Rev. 89:49-68), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPS V promoter, the tetracycline-inducible CMV promoter (such as the human immediate-early CMV promoter), the constitutive CMV promoter, the EF1 alpha promoter, the phosphoglycerate kinase (PGK) promoter and promoter-enhancer combinations known in the art.

In some embodiments, the vector(s) comprises an EF1 alpha promoter. In certain embodiments, the vector(s) is a lentiviral vector comprising an EF1 alpha promoter. For example, the vector(s) may comprise a EF1a promoter-modified pUltra lentiviral vector, which is commercially available from Addgene (Watertown, MA, USA).

### Method of Modifying a Treg

The present invention also provides a method of modifying a Treg, the method comprising genetically engineering a Treg to express:
a) a non-activating site-targeting receptor or ligand; and
b) a reduced expression level of FAS.

Optionally, the method also comprises genetically engineering the Treg to express a chemokine receptor or a functional variant thereof, as disclosed above.

The genetic engineering of Tregs can be carried out according to standard cloning and expression techniques, which are known in the art (e.g., as described in Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989).

The method may comprise introducing the vector(s) and/or nucleotide sequence(s) of the invention into the Treg. In some embodiments, the method comprises transfecting the Treg with the vector(s) and/or nucleotide sequence(s) of the invention to express the non-activating site-targeting receptor or ligand and a reduced expression level of FAS.

The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like.

Alternatively, the method may comprise transducing the Treg with the vector(s) and/or nucleotide sequence(s) of the invention to express the non-activating site-targeting receptor or ligand and a reduced expression level of FAS. For example, a viral vector, as disclosed above, may be used for delivery of the nucleotide sequence(s) and/or vector(s).

In some embodiments, the method comprises:
i) activating the Treg; and
ii) genetically engineering the Treg to express a) a non-activating site-targeting receptor or ligand;
   b) a reduced expression level of FAS; and optionally
   c) a chemokine receptor or a functional variant thereof.

Activation of the Treg may be prior to, simultaneous to or after genetically engineering the Treg. Activation of the Treg may be prior to genetically engineering the Treg. In some embodiments, the method comprises a simultaneous activation and genetic engineering step. Activation of the Treg may comprise incubation of the Treg with anti CD3 and anti-CD28 coated beads or with PMA and ionomycin or rapamycin. Other methods for the activation of Tregs will be well known to those skilled in the art.

In some embodiments, the method further comprises step iii) of expanding the Tregs after genetically engineering the Tregs. Expansion may comprise incubation in a media comprising IL-2. In some embodiments, expansion may comprise incubation in a puromycin-comprising media. In such embodiments, when the Tregs have been genetically engineered to express PAC, this ensures that only successfully genetically engineered Tregs remain viable. Thus, any non-genetically engineered cells are eliminated from the expansion step, thereby improving the purity of the resulting cell population.

### Pharmaceutical Compositions

Also provided is a pharmaceutical composition comprising the Treg, polynucleotide sequence(s) and/or the vector(s) according to the invention and a pharmaceutically or physiologically acceptable diluent and/or carrier.

The carrier and/or diluent is generally selected to be suitable for the intended mode of administration and can include agents for modifying, maintaining, or preserving, for example, the pH, osmolarity, viscosity, clarity, colour, isotonicity, odour, sterility, stability, rate of dissolution or release, adsorption, or penetration of the composition. Typically, these carriers and/or diluents include aqueous or alcoholic/aqueous solutions, emulsions, or suspensions, including saline and/or buffered media.

Suitable further agents for inclusion in the pharmaceutical composition include, but are not limited to, amino acids (such as glycine, glutamine, asparagine, arginine, or lysine), antimicrobials, antioxidants (such as ascorbic acid, sodium sulphite, or sodium hydrogen-sulphite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as free serum albumin, gelatin, or immunoglobulins), colouring, flavouring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as Polysorbate 20 or Polysorbate 80; Triton; tromethamine; lecithin; cholesterol or tyloxapal), stability enhancing agents (such as sucrose or sorbitol), tonicity enhancing agents (such as alkali metal halides, such as sodium or potassium chloride, or mannitol sorbitol), delivery vehicles, excipients and/or pharmaceutical adjuvants.

The carrier and/or diluent may be a parenteral, optionally intravenous vehicle. Suitable parenteral vehicles include sodium chloride solution, Ringer's dextrose, dextrose and sodium chloride and lactated Ringer's. Suitable physiologically acceptable thickeners such as carboxymethylcellulose, polyvinylpyrrolidone, gelatin and alginates may be included. Intravenous vehicles include fluid and nutrient replenishers and electrolyte replenishers, such as those based on Ringer's dextrose. In some cases, one might include agents to adjust tonicity of the composition, for example, sugars, polyalcohols such as mannitol, sorbitol, or sodium chloride in a pharmaceutical composition. For example, in many cases it is desirable that the composition is substantially isotonic. Preservatives and other additives, such as antimicrobials, antioxidants, chelating agents, and inert gases, may also be present. The precise formulation will depend on the route of administration. Additional relevant principle, methods and components for pharmaceutical formulations are well known (see, e.g., Allen, Loyd V. Ed, (2012) Remington's Pharmaceutical Sciences, 22^{nd} Edition).

A pharmaceutical composition of the present invention can be administered by one or more routes of administration using one or more of a variety of methods known in the art. As will be appreciated by the skilled person, the route and/or mode of administration will vary depending upon the desired results. Routes of administration for pharmaceutical compositions of the invention include intravenous, intramuscular, intradermal, intraperitoneal, intrapleural, subcutaneous, intratumoural, spinal, intra-bone marrow or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intratumoural, intrapleural and intra-sternal injection and infusion. In some embodiments, the pharmaceutical composition is administered intravenously. In other embodiments, administration is intrapleural or intraperitoneal. When parenteral administration is contemplated, the pharmaceutical compositions are usually in the form of a sterile, pyrogen-free, parenterally acceptable composition. A particularly suitable vehicle for parenteral injection is a sterile, isotonic solution, properly preserved. The pharmaceutical composition can be in the form of a lyophilizate, such as a lyophilized cake.

Alternatively, the pharmaceutical composition of the invention can be administered by a nonparenteral route, such as a topical, epidermal, or mucosal route of administration, for example, intranasally, orally, vaginally, rectally, sublingually, or topically.

In some embodiments, the pharmaceutical composition is for subcutaneous administration. Typically, the pharmaceutical compositions for subcutaneous administration contain suitable stabilizers (e.g., amino acids, such as methionine, and or saccharides such as sucrose), buffering agents and tonicifying agents. Alternatively, the pharmaceutical composition may be for intra-bone marrow administration.

### Kit

The invention also provides a kit comprising the genetically engineered Treg, polynucleotide(s) and/or vector(s) of the invention. The kit may further comprise instructions for use. In some embodiments, the genetically engineered Treg, polynucleotide(s) and/or vector(s) is provided in an aqueous solution, optionally buffered solution and/or at a temperature of at least −20° C.

### Methods of Treatment

Also provided is a method of treating or preventing a disease in a subject, wherein the method comprises administering to the subject the genetically engineered Treg, polynucleotide(s), vector(s), or the pharmaceutical composition of the invention.

Preferably, the method comprises administering to the subject a population of genetically engineered Tregs, as described above.

The method typically comprises administering a therapeutically effective amount or a prophylactically effective amount of the genetically engineered Treg, polynucleotide(s), vector(s), or the pharmaceutical composition of the invention. A therapeutically effective amount is an amount which ameliorates one or more symptoms, such as all the symptoms, of the disease and/or abolishes one or more symptoms, such as all the symptoms, of the disease. The therapeutically effective amount preferably cures the disease. A prophylactically effective amount is an amount which prevents the onset of the disease and/or prevents the onset of one or more symptoms, such as all the symptoms, of the disease. The prophylactically effective amount preferably prevents the subject from developing the disease. Suitable amounts are discussed in more detail below.

The genetically engineered Treg, polynucleotide(s), vector(s), or the pharmaceutical composition of the invention may be administered to a subject that displays symptoms of disease. The genetically engineered Treg, polynucleotide(s), vector(s), or the pharmaceutical composition of the invention may be administered to a subject that is asymptomatic, i.e., does not display symptoms of disease. The genetically engineered Treg, polynucleotide(s), vector(s), or the pharmaceutical composition of the invention may be administered when the subject's disease status is unknown, or the subject is expected not to have a disease. The genetically engineered Treg, polynucleotide(s), vector(s), or the pharmaceutical composition of the invention may be administered to a subject that is predisposed, such as genetically predisposed, to developing the disease.

The subject may be a mammal. Optionally, the subject is a human, horse, dog or cat. In some embodiments, the subject is human. Alternatively, the subject may be a horse.

In embodiments where the subject is human, the subject may be a human adult or child. In the context of the present invention, an adult will be understood to be an at least 18-year-old human. A child will be understood to be a human less than 18 years old. In some embodiments, the adult is at least 60 years old.

Various diseases are suitable for treatment or prophylaxis by administration of the genetically engineered Treg, polynucleotide(s), vector(s), or pharmaceutical composition of the invention. Any disease which can be treated or prevented using immunotherapy is envisaged. In particular, inflammatory disease, such as autoimmune disease or infection, is envisaged.

In the context of the present invention, an inflammatory disease is a disease or infection which comprises the damage or destruction of healthy viable cells. Examples of inflammatory diseases include, but are not necessarily limited to autoimmune disease, allergy, asthma, coeliac disease, nephritis, hepatitis, reperfusion injury, graft versus host disease (GvHD), transplant rejection and infection. By “infection”, this will be understood to bacterial or viral infection.

In some embodiments, the disease comprises an autoimmune disease, viral infection or cancer.

Autoimmune disease may comprise rheumatoid arthritis, psoriasis, system lupus erythematosus (lupus), inflammatory bowel disease, multiple sclerosis, diabetes, Guillain-Barre syndrome, chronic inflammatory demyelinating polyneuropathy, Graves' disease, Hashimoto's thyroiditis, Myasthenia gravis, Aplastic Anaemia (AA), Vasculitis or combinations thereof.

In some embodiments, the disease is selected from rheumatoid arthritis, lupus, multiple sclerosis, diabetes, aplastic anaemia (AA) and cancer.

In some embodiments, the disease comprises or consists of Aplastic anaemia (AA). Aplastic anaemia (AA) is a condition characterized by failure of the bone marrow and stem/progenitor cells to produce all types of blood cells (pancytopenia).

Causes of aplastic anaemia can vary and are not always known. The most common cause of AA is autoimmune, comprising immune mediated destruction of bone marrow stem/progenitor cells and diminished wild type anti-inflammatory Tregs. This may be referred to as immune AA (iAA). Other causes can include injury of the bone marrow, for example from radiation and chemotherapy treatments, exposure to toxic chemicals, use of certain drugs, such as certain rheumatoid arthritis medication, viral infection and pregnancy. In some instances, the cause of AA is unknown, which may otherwise be referred to as idiopathic aplastic anaemia.

In some embodiments, the AA comprises or consists of idiopathic AA or immune AA. Preferably, the AA comprises or consists of immune AA.

Without wishing to be bound by theory, the present inventors believe that the genetically engineered Tregs of the present invention may be especially useful for the treatment of AA, particularly immune AA. Treg numbers in AA, particularly immune AA, can be lower than for healthy donors, which can make it difficult to obtain sufficient numbers of Tregs (especially autologous Tregs) for cell therapy. The targeted and apoptosis-resistant nature of the Tregs of the present invention may advantageously reduce the number of Tregs required for effective cell therapy.

Symptoms of AA may include, but are not necessarily limited to fatigue, shortness of breath, rapid or irregular heart rate, pale skin, frequent or prolonged infections, unexplained or easy bruising, nosebleeds and bleeding gums, prolonged bleeding from cuts, skin rash, dizziness, headache and fever.

As described above, the genetically engineered Treg of the invention expresses a non-activating site-targeting receptor or ligand. Preferably, the site to be targeted comprises a site specific to the disease to be treated. For example, if the disease comprises cancer, the non-activating site-targeting receptor or ligand may comprise a non-activating tumour microenvironment (TME)-targeting receptor or ligand. In embodiments where the disease comprises an autoimmune disease, the non-activating site-targeting receptor or ligand may comprise a non-activating autoimmune disease site-targeting receptor or ligand. For example, where the disease comprises MS, the non-activating site-targeting receptor or ligand may comprise a non-activating CNS-targeting receptor or ligand.

In embodiments where the disease comprises or consists of AA, the non-activating site-targeting receptor or ligand preferably comprises a non-activating bone marrow-targeting receptor or ligand. More preferably, where the disease comprises or consists of AA, the non the non-activating site-targeting receptor or ligand comprises a non-activating CD34-targeting receptor or ligand, as described above.

The present inventors believe that by linking the target site to a site of the disease to be treated, the genetically engineered Tregs of the present invention have improved efficacy in the treatment of the disease. The improved efficacy may be due to improved migration of the Tregs to the site of the disease. By ensuring that the receptor or ligand is non-activating, when the Treg reaches the site, the receptor or ligand facilitates the migration of the Treg to the site without activation of the Treg, which may reduce death of the Treg and in turn increase viability of the Treg.

Administration of the genetically engineered Treg, polynucleotide(s), vector(s), or the pharmaceutical composition of the invention to the subject may reduce symptoms by at least about 10%, about 20%, about 30%, about 40%, about 50%, about 60%, about 70%, about 80%, about 90%, or even about 100%, when compared to an untreated subject.

In embodiments comprising a population of genetically engineered Tregs, the number of genetically engineered Tregs administered to the subject should take into account the route of administration, the disease being treated, the weight of the subject and/or the age of the subject. In general, from about 1×10⁶ to about 1×10¹¹ genetically engineered Tregs may be administered to the subject. In some embodiments, from about 1×10⁶ to about 1×10⁹ genetically engineered Tregs, or from about 1×10⁸ to about 1×10⁹ genetically engineered Tregs are administered to the subject.

The invention also provides the genetically engineered Treg, polynucleotide sequence(s), vector(s), or the pharmaceutical composition of the invention for use in any of the therapeutic methods described above. Thus, also provided is the genetically engineered Treg, polynucleotide sequence(s), vector(s), or the pharmaceutical composition of the invention for use in the treatment or prevention of a disease. In particular, the invention provides the genetically engineered Treg, polynucleotide sequence(s), vector(s), or the pharmaceutical composition of the invention for use in the treatment or prevention of a disease selected from rheumatoid arthritis, lupus, multiple sclerosis, diabetes, aplastic anaemia (AA) and cancer.

Preferably, the genetically engineered Treg, polynucleotide sequence(s), vector(s) or the pharmaceutical composition of the invention is for use in the treatment or prevention of Aplastic Anaemia (AA).

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application, it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### EXAMPLES

### Materials and Methods

### Cloning of Lentiviral Transfer Plasmids, Transformation, Maxiprep and Lentiviral Production

Plasmids from Addgene were used as templates to prepare the inserts of the sequences of interest by PCR using Q5 Hot Start High-Fidelity DNA Polymerase (New England Biolabs) according to manufacturer's instructions. LentiCRISPRv2 was used to amplify the Puromycin resistant gene insert using the Forward primer 5′ gtcgtgaaaactacccctaaaagctagccgccaccatgaccgagtacaagcccacgg 3′ (SEQ ID NO: 20) and the reserve primer 5′ ccccttccttctccggatccggcaccgggcttgcgg3′ (SEQ ID NO: 21).

miR30a fragments were generated by mixing complementary single-stranded (ss) DNA oligonucleotides (Integrated DNA Technologies), which were then used as templates to create two miRshRNA inserts for each construct. The first insert, termed insert A, used the forward primer 5′cgagctgtacaagtgatcagaatttgtttgaatgaggcttcagtactttac3′ (SEQ ID NO: 22) and the reverse primer 5′tacatctgtggcttcactatttaatcaatgtgtcatacgcgcgctcactgtcaacagc3′ (SEQ ID NO: 23) for miRshFAS1. The same forward primer (SEQ ID NO: 22) was used for miRshFAS2 and for miRshGFP. For miRshFAS2, the reverse primer 5′tacatctgtggcttcactaaagtttggtttacatctgcacgcgctcactgtcaacagc3′ (SEQ ID NO: 24) was used.

For miRshGFP, the reverse primer 5′tacatctgtggcttcactatgaacttcagggtcagcttgctcgctcactgtcaacagc3′ (SEQ ID NO: 25) was used.

For insert B, the forward primer for miRshFAS1 5′ was 5′tagtgaagccacagatgtatttaatcaatgtgtcatacgcttgcctactgcctcgg3′ (SEQ ID NO: 26). The forward primer for insert B of miRshFAS2 was 5′tagtgaagccacagatgtaaagtttggtttacatctgcacttgcctactgcctcgg3′ (SEQ ID NO: 27) and the forward primer for insert B of miRshGFP was 5′ tagtgaagccacagatgtatgaacttcagggtcagcttgcctgcctactgcctcgg3′ (SEQ ID NO: 28). The reverse primer was the same for all three insert B's (5′ cgacgactccggaacgaattaaaaaagtgatttaatttataccattttaattcagc3′ (SEQ ID NO: 29)). All primers were from IDT. Cloning was conducted using NEBuilder HiFi DNA Assembly (New England Biolabs) using 20 ng backbone, pUltra previously modified to express TdTomato as a reported, and mass of insert equivalent to a 1:1 molar ratio.

For the QBEND/10 lentiviral plasmid, the QBEND/10 gblocks (IDT) designed on SnapGene software (www.snapgene.com), were inserted into the previous generated plasmids directly as a fragment using NEBuilder HiFi DNA Assembly (New England Biolabs). Subsequent QBEND/10 plasmids were derived from modifying the linker sequence between the light and heavy chain, also designed as gBlocks on SnapGene software (www.snapgene.com) and cloned using NEBuilder HiFi DNA Assembly (New England Biolabs).

For the CXCR4 lentiviral plasmid, the CXCR4 gblock (IDT) designed on SnapGene software (www.snapgene.com), were inserted into the previous generated plasmids directly as a fragment using NEBuilder HiFi DNA Assembly (New England Biolabs).

NEBuilder HiFi DNA Assembly reactions were transformed by mixing with NEB 5-alpha competent E. coli (New England Biolabs), according to the manufacturer's instructions and bacteria was then plated on agar plates containing ampicillin and incubated overnight. The resulted colonies were screened by analytical restriction digestion and sanger sequencing. Colonies were then grown and miniprepped to isolate the plasmid DNA with the E.Z.N.A Plasmid DNA Mini Kit II (Omega Bio-Tek).

2^{nd} Generation Lentivirus was produced by transfecting HEK-293T cells with the purified DNA plasmids, in combination with the packaging plasmids, pVSV-G and pCRV-1, and polyethylenimine (Sigma). 48 h and 72 h after transfection, media containing virus was collected and pooled. Lentivirus was then concentrated by high-speed centrifugation and stored at −80 C until used.

### In Vitro Treg Isolation, Expansion and Transduction

Human Tregs were magnetically isolated from total peripheral blood mononuclear cells (PBMCs) using CD4⁺CD25⁺ Regulatory T Cell Isolation Kit, human (Miltenyi) according to the manufacturer's instructions. In summary, non-CD4⁺ cells were depleted, followed by a positive selection of CD25⁺ cells, using antibodies conjugated with Microbeads. CD4⁺CD25⁻ cells were also collected and frozen until needed.

Isolated Tregs were then cultured in Prime XV T cell expansion XSFM (Irvine Scientific), supplemented with 5% human AB serum (Sigma Aldrich) in the presence of 100 nM rapamycin (LC Laboratories) for 4 to 6 weeks. Tregs were stimulated with Dynabeads human T-activator CD3/CD28 (cell:bead ratio=1:1; Thermo Fisher Scientific) and 1000 IU/ml of human IL-2 (Proleukin; Novartis).

24 h after activation, 50 μl of concentrated lentivirus was added to the culture and 96 h after, 1 μg/mL of Puromycin (Sigma) was also added to remove untransduced cells. The culture was replenished every 2 days with human IL-2 and every week with fresh culture media and Dynabeads human T-activator CD3/CD28.

### Flow Cytometry

Anti-CD4-BUV395 (BD Biosciences), anti-CD25-PE-Cy7 (Biolegend), anti-CD127-BV650 (Invitrogen) and anti-CD95 conjugated to PerCP-Cy5.5 (Biolegend) were used for surface staining. Anti-human Foxp3 conjugated with APC was used for intracellular staining after fixation and permeabilization according to the manufacturer's instructions (eBioscience). For detection of QBEND/10 expression, a two-step staining was performed after Fc receptor blocking (TruStain FcX, Biolegend) using human recombinant CD34 Fc-Tagged (Stratech Scientific Ltd) followed by anti-Human-IgG conjugated to Alexa Fluor 647 (Biolegend). For detection of the Myc-tag reporter mouse anti-c-Myc (produced in-house) was used, followed by APC tagged anti-mouse IgG (Biolegend).

Analysis was performed on a BD FACS Fortessa (BD Biosciences) and FlowJo (BD Biosciences), where cells were gated based on the expression of the reporter gene TdTomato.

### Suppression Assay

Effector T cells were stained with a fluorescent proliferation dye, cell trace violet (Invitrogen) and Tregs with carboxyfluorescein diacetate succinimidyl ester (CFSE, Invitrogen) and cocultured with autologous Tregs at different Tcon:Treg ratios (16:1, 8:1, 4:1, 2:1, and 1:1) for 5 days in the presence of anti-CD3/CD28 beads (Gibco, Tcon:beads ratio=20:1). Cells were harvested after 5 days, stained with Fixable Viability Dye eFluor 780 (Thermo Fisher Scientific) and analysed on a BD FACS Fortessa.

### FasL-Induced Apoptosis Assay

Tregs were stimulated with 5 μg/mL of anti-Fas (Millipore) for 5 hours and stained with Fixable Viability Dye eFluor 780 (Thermo Fisher Scientific) followed by Annexin V APC (BioLegend) in binding buffer (BioLegend). Stained cells were analysed on a BD FACS Fortessa and FlowJo, where percentages of early (Viability Dye eFluor 780-Annexin V+) and late (Viability Dye eFluor 780+ Annexin V+) apoptotic cells were calculated in the TdTomato positive fraction.

### Western Blotting

Detection of BCL-2 and STAT5 was done by Western blotting and protein expression levels were quantified by Image Studio™ Lite Version 5 software (LI-COR Biosciences) against loading control β-ACTIN according to the band intensity.

### Xenotransplantation and Imaging

NOD/SCID/IL2rγ−/−/IL-3/GMCSF/SCF (NSG-SGM3) mice were obtained from Leonard Shultz (The Jackson Laboratory) and bred at the Francis Crick Institute biological resources facility. All animal experiments were performed in accordance with UK Home Office and Francis Crick guidelines. PBMCs and Tregs were either coinjected or injected alone into the recipient mice via the IV route. PBMCs and Tregs were coinjected at a 1:1 ratio. Mice were euthanized either when animals lost 20% of body weight or at week 12. Then, mouse tissues were recovered and analysed by Flow Cytometry. Engraftment was assessed when mice either lost 20% of weight or at the end of experiment (up to 18 weeks), were imaged using the Xenogen IVIS imaging system following D-luciferin (Caliper Life Sciences) injections. D-luciferin was injected via the intra-peritoneal (150 mg/kg) route. Bioluminescence images were taken from dorsal side of the mice. The photons emitted from luciferase-expressing Tregs cells, expressed as Flux (photons/second/cm²/steradian) were quantified and analysed using the ‘Living image’ software (Caliper Life Sciences).

Harvested bones and other soft tissues from mice were recovered and fixed overnight in 10% neutral buffered formalin. Bones were then decalcified with Osteosoft (Millipore) for 7 days. All the other tissues were processed, paraffin embedded and then sectioned (5 μm) for histological studies. Following on, H&E (Haemotoxylin and Eosin) staining was performed first to assess quality of the sections and to analyse the tissue sections under the microscope. Tissue images were taken using Zeiss Axio Scan. Z1 slice scanner using Zen blue edition software. Data from images was obtained using Fiji software equipped with both grid overlay and Cell Counter plugins.

### Statistical Analysis

Statistical analysis was performed using Prism Version 7 software (GraphPad Software). Statistical significance was calculated by P value using unpaired Student t test. A P value<0.05 was considered statistically significant.

### Example 1: Death Receptor Signalling Drives Treg Sub-Population Depletion in Aplastic Anaemia (AA)

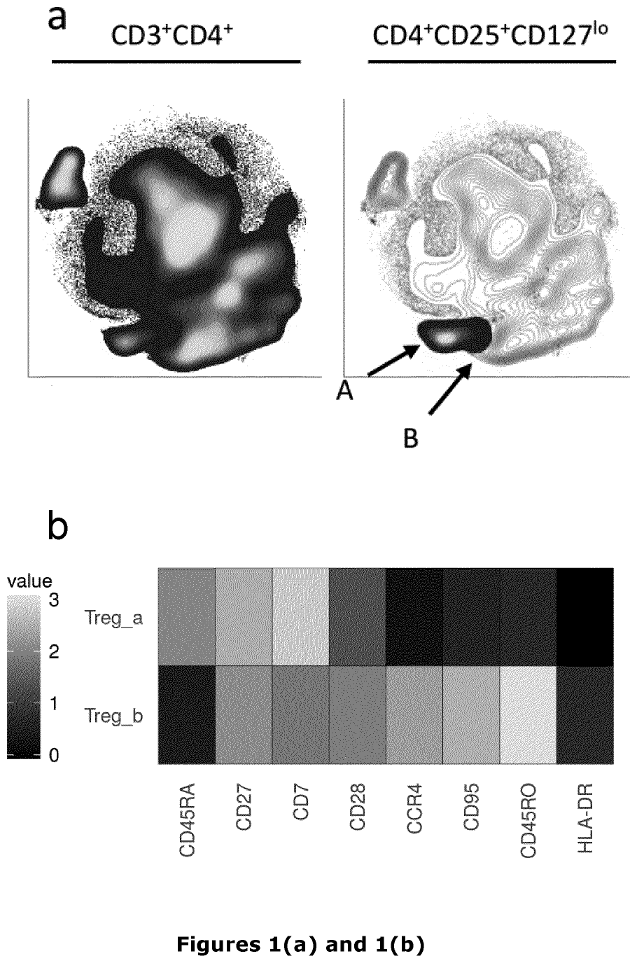

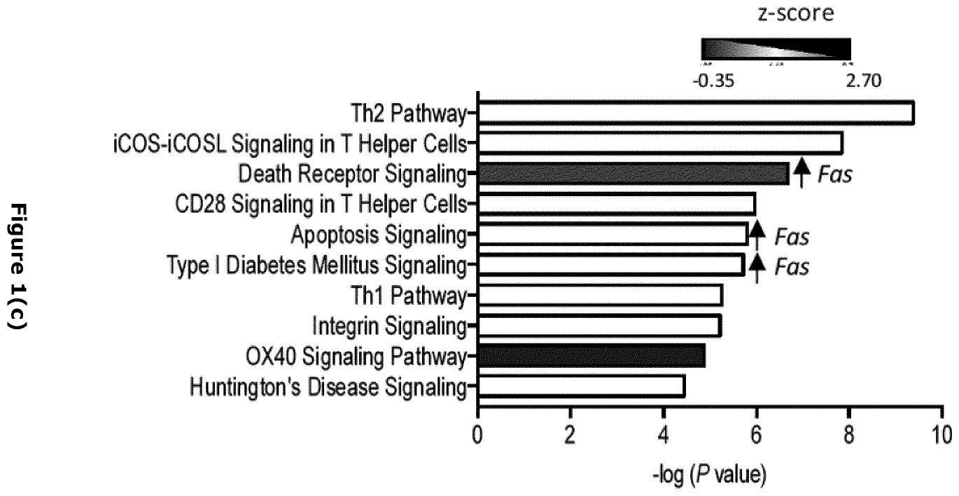

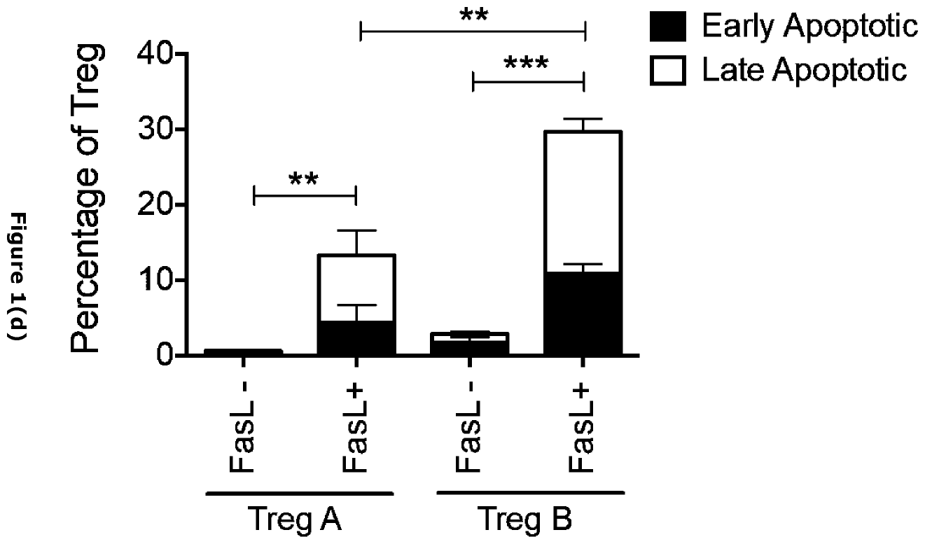

Using deep phenotyping, we have identified an immunological signature in AA that predicts clinical response to IST (Kordasti et al.). This signature includes two novel subpopulations of Tregs that we termed Tregs A and B (FIG. 1a). The more functional and proliferative Treg subpopulation, Treg B, expresses high levels of FAS (CD95), which makes them susceptible to FAS-L mediated cell death (FIG. 1b). This population was profoundly deficient in the bone marrow of subjects with AA.

By comparing gene expression profiles of AA Treg-B to healthy donors (HD) Treg-B we also found that the top significant upregulated pathways in AA Treg-B were apoptosis-related pathways (−log P value=4.69; z score=1.414, FIG. 1c) (Lim et al.). In in vitro assays we have demonstrated that FAS-L induces significantly higher early and late apoptosis in FAS⁺ Treg B compared to Treg A subpopulation⁷ which further contributes to immune dysregulation (FIG. 1d).

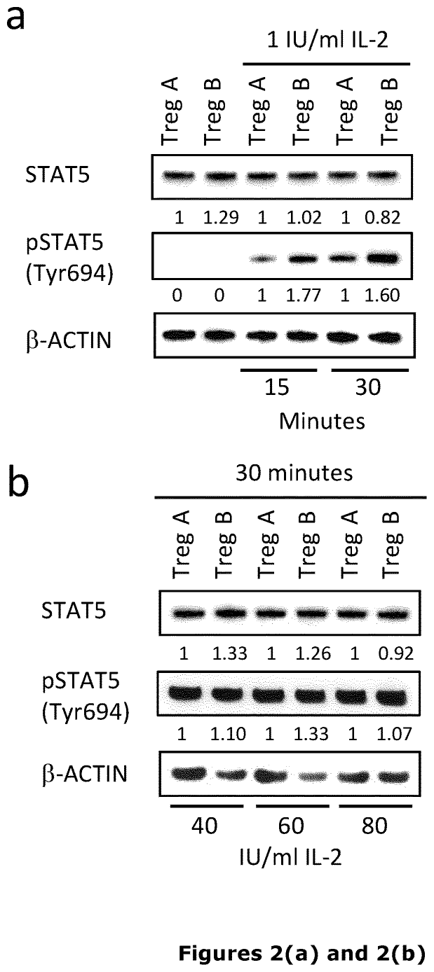

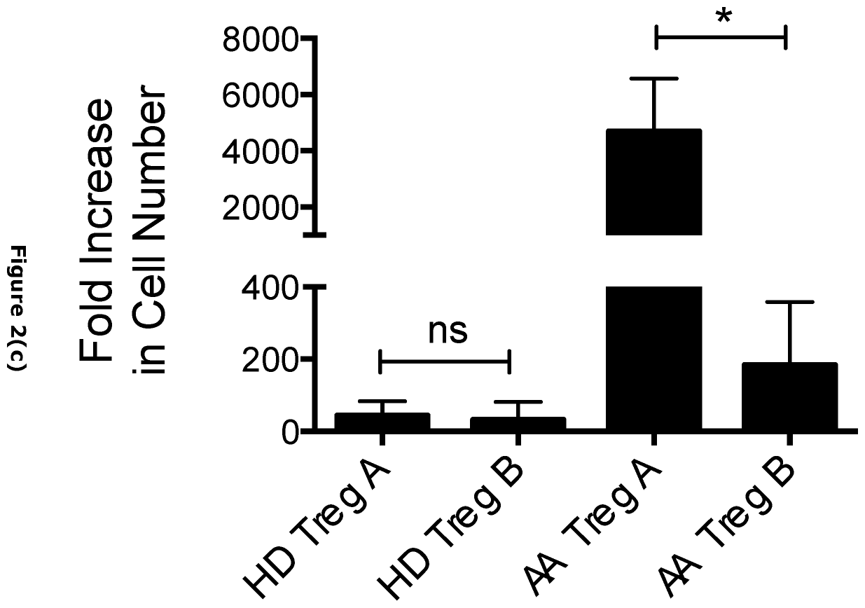

It was not clear why Treg A, which are more resistant to Fas-L, cannot expand. To address this, we tested the “IL-2 responsiveness” of Treg A in vitro. When Treg A and B were treated with low concentration of IL-2 (1 IU/mL) for 15 and 30 minutes, lower pSTAT5 protein expression was observed in Treg A compared to Treg-B at 15 minutes, suggesting a delay in response to IL-2 (FIG. 2A). Nevertheless, when IL-2 concentration was increased to 40, 60 and 80 IU/mL, protein expression of pSTAT5 in Treg A increased and was comparable to Treg-B after 30 minutes of IL-2 exposure (FIG. 2B) (Kordasti et al.). These data suggest that Treg A are less responsive to low concentrations of IL-2 compared to Tregs B but respond equally well to higher concentrations of IL-2 which could be due to a relatively lower expression of CD25.

### Example 2: Treg from HD and AA Subjects can be Robustly Expanded Ex Vivo and Retain Suppressive Capacity

There is considerable interest in Tregs as cell therapy products since they can be expanded ex vivo and infused into subjects. We tested the in vitro expandability of AA Tregs in a Treg-promoting culture condition, which comprised a Prime-XV T Cell Expansion XSFM (Irvine Scientific, 91141) supplemented with 5% AB serum and 100 nM Rapamycin (LC Laboratories, R-5000) with high concentration of IL-2, 1000 U/mL, (Proleukin, Novartis, CLB-P-476-750-14002_GB) and 1:1 Dynabeads Human T-Activator (Gibco, 11132D) for cell activation. Both Treg-A and B isolated from HD and AA subjects expanded for up to 4 weeks. Surprisingly, for AA subjects, Treg-A expanded at a significantly higher rate than Treg-B (P value=0.0198), with an average 4708-fold increase (range, 3388.7-6027.4) compared to 185-fold (range, 32.39-374) (FIG. 2C) (Lim et al.). TSDR10 cytosine guanine dinucleotide sites in the expanded Treg-A and B from both HD as well as AA subjects were highly unmethylated compared to the non-Treg (CD4⁺CD25^{lo}CD127^{hi}) population suggesting that expanded Tregs have a stable FoxP3 expression. Functionally, both HD and AA expanded Tregs were equally able to suppress the proliferation of conventional T cells (T_{con}), (FIG. 2D). Similarly, both expanded Treg-A and B from AA subjects were equally able to suppress the proliferation of T_{con} cells.

Treg possess some degree of plasticity. Hence, we studied the plasticity of expanded Tregs by culturing them in the presence of IL-1β and IL-6 to promote IL-17A secretion. No or negligible increases in IL-17A expression in the expanded Treg-A and B were observed in contrast to control CD4⁺CD25⁺ cells. The expanded Treg retain their phenotype in the face of cytokine challenge.

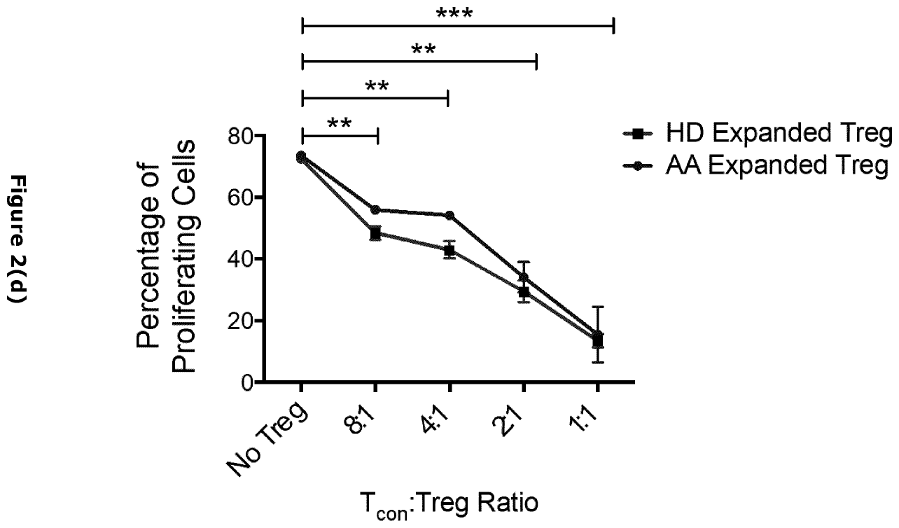

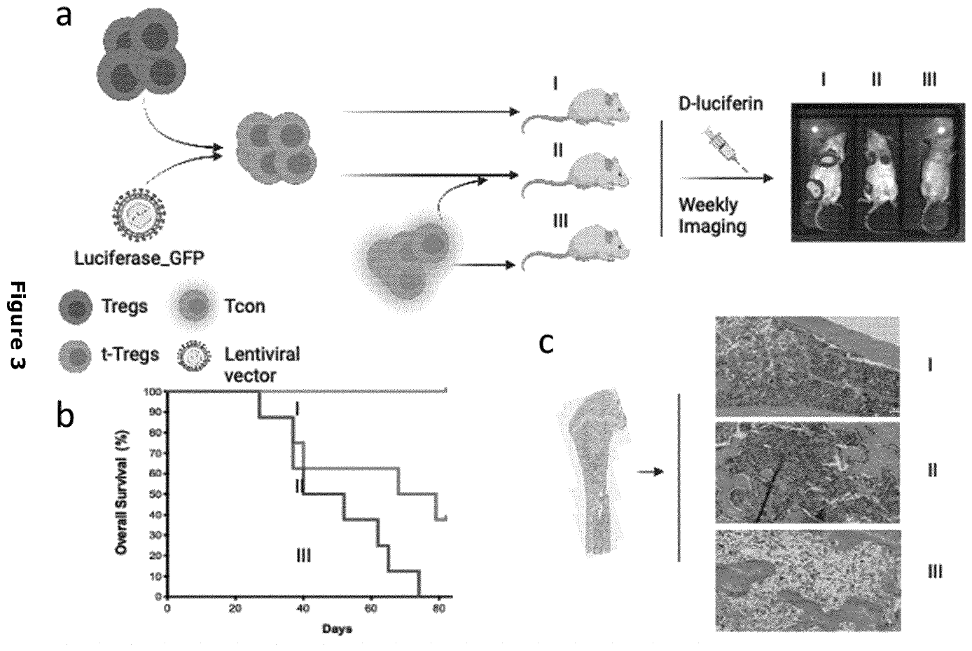

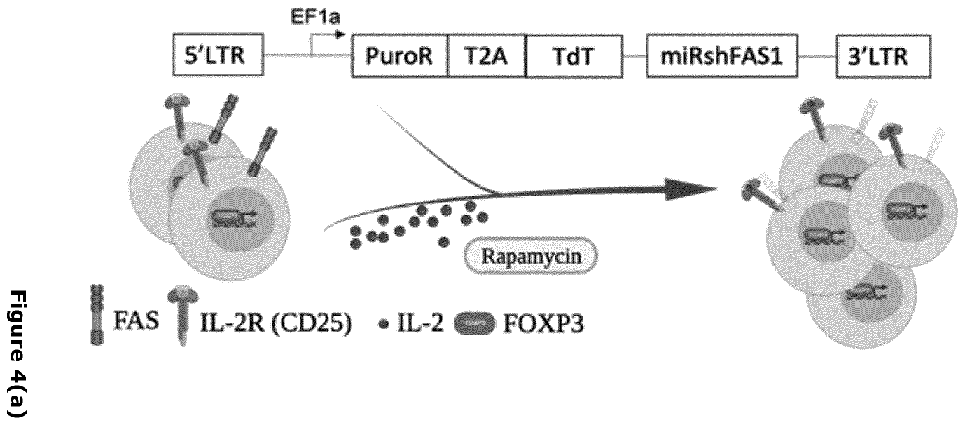

To evaluate the suppressive ability of the expanded Tregs in vivo, we used NOD/SCID/IL2rγ^{-/-}/IL-3/GM/SF (NSG-SGM3) humanized mice. The NSG-SGM3 mouse model efficiently supports the development and maintenance of human Tregs. AA Tregs were expanded for 4 weeks with anti-CD3/CD28 and high dose IL-2 and transduced with luciferase-GFP, using lentiviral vector. Tregs remain detectable by imaging for up to 3 weeks when injected alone, and up to 6 weeks when injected with Tcon (FIG. 3A).

### Example 3: Generation of Tregs Genetically Engineered to Express a Reduced Expression Level of FAS

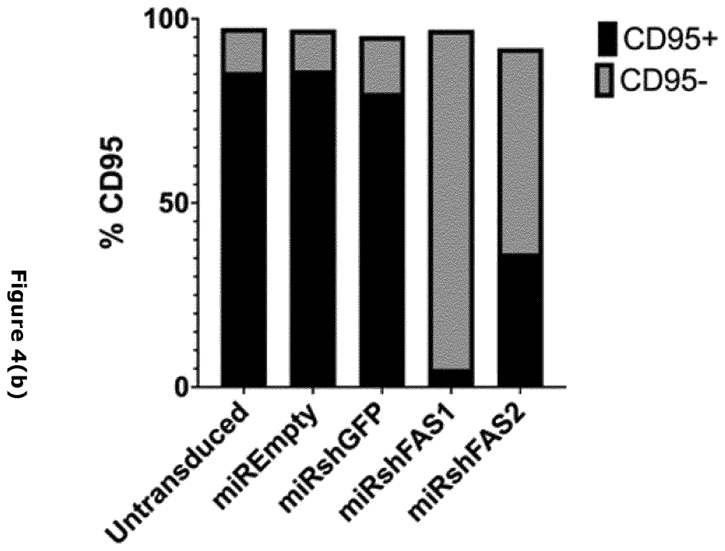

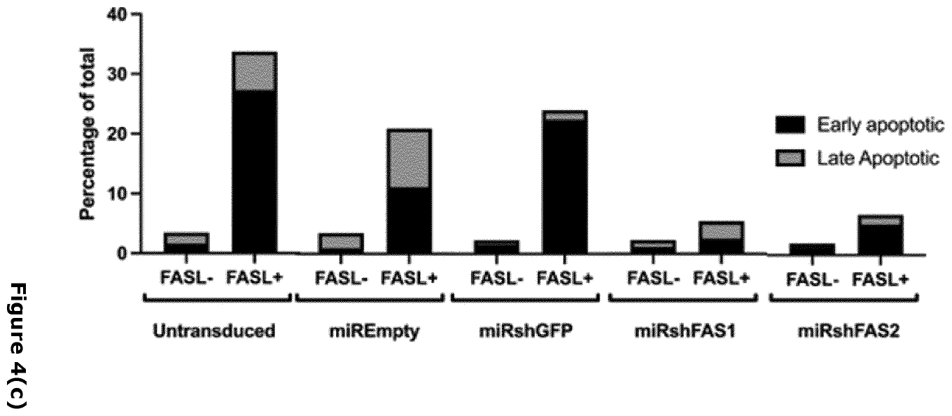

We have found that the expression level of FAS can be reduced in Treg cells by genetically engineering Tregs, which increases the resistance of the genetically engineered Tregs to apoptosis. This may otherwise be referred to as FAS knockdown (KD). To reduce the expression level of FAS, human Tregs were transduced with a lentiviral vector comprising an EF1α promoter driven miR30-shRNA specific for a target region of the FAS gene. The lentiviral vector comprised a modified pUltra backbone. A puromycin resistant gene was also introduced to the vector to eliminate non-transduced cells during culture, preventing their expansion and improving the purity of genetically engineered Tregs. FIG. 4A is a schematic to illustrate the content of the lentiviral vector and the genetic engineering process.

Two plasmids were generated, with different miR30-shRNA sequences, targeting FAS (miRshFAS1 and miRshFAS2), and two controls. miRshFAS1 was encoded by SEQ ID NO: 9, while miRshFAS2 was encoded by SEQ ID NO: 10. The shFAS1 sequence comprised SEQ ID NO: 1, while the shFAS2 sequence comprised SEQ ID NO: 2. The 2^{nd} generation lentiviruses, produced after HEK293T cell transfection, were used to deliver these plasmids to proliferating Tregs, stimulated with anti-CD3/CD28, high dose IL-2 and Rapamycin.

Both FAS targeting shRNAs were able to reduce the expression of FAS on Tregs. Of the two shRNAs, miRshFAS1 resulted in a 90% reduction in FAS expression and miRshFAS2 a 60% reduction in FAS expression. Thus, in some instances only 10% of the transduced cells remained CD95⁺, with FAS expression in the remaining cells reduced to be undetectable. The resulting Treg cultures demonstrated stable reductions in FAS over 15 days of expansion (FIG. 4B). The genetically engineered Tregs were resistant to FAS-L induced apoptosis (FIG. 4C), were able to expand further at a rate comparable to unmodified Tregs (FIG. 4D) and remained able to suppress T_{con} proliferation in suppression assays (FIG. 4E). This confirmed that the genetically modified Tregs maintained their suppressive functionality and could expand.

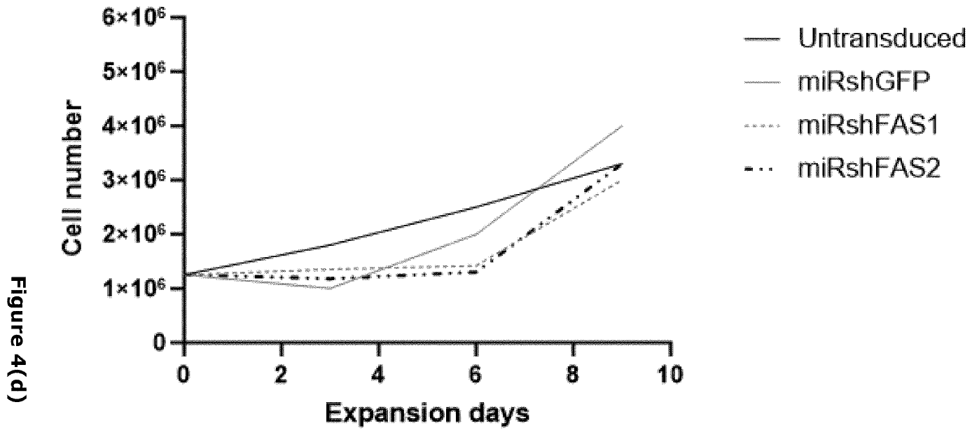

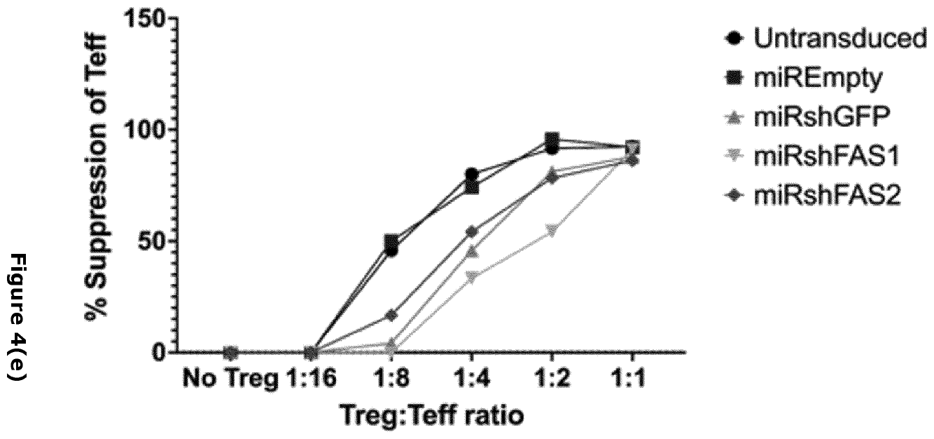

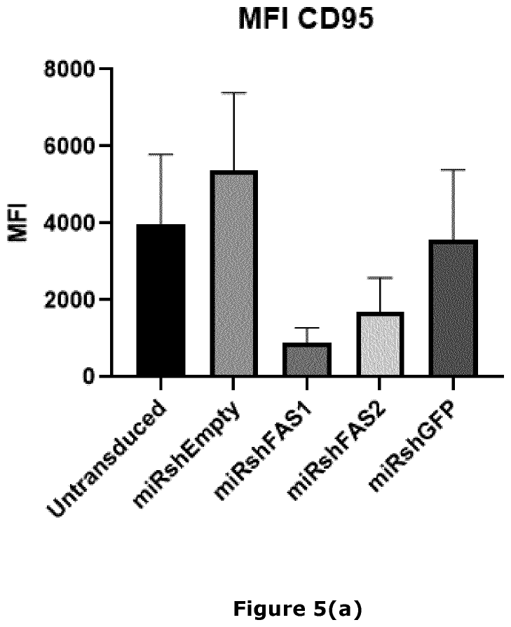

The ability of the FAS targeting miRshRNAs to reduce FAS expression was further explored. We decided to specifically investigate the MFI values of FAS in healthy donor Tregs transduced with miRshFAS1, miRshFAS2, a GFP negative control (miRshGFP), an empty vector control (miRshEmpty) and untransduced Tregs as a further control. The results are shown in FIG. 5A. In Tregs not comprising miRshFAS1 or miRshFAS2, FAS MFI varied between about 3000 and 6000. However, in miRshFAS1 and miRshFAS2 transduced cells, the MFI was reduced considerably to between about 1000 and 2000. This represents a reduction in MFI of between about 50 and 80%.

It was then decided to investigate the stability of the FAS knockdown. To assess this, FAS (or CD95) MFI was measured four and six weeks after the Tregs were transduced with miRshFAS, miRshGFP or untransduced controls. The results are shown in FIG. 5B. These confirm that the FAS knockdown is stable, persisting for at least six weeks.

### Example 4: Generation of Tregs Genetically Engineered to Express a Non-Activating Site-Targeting Receptor or Ligand and a Reduced Expression Level of FAS

To direct Tregs to bone marrow to deliver functional suppression of inflammation with lower Treg numbers, we further engineered Tregs from healthy donors to simultaneously express a non-activating CD34-targeting single chain variable fragment (scFv).

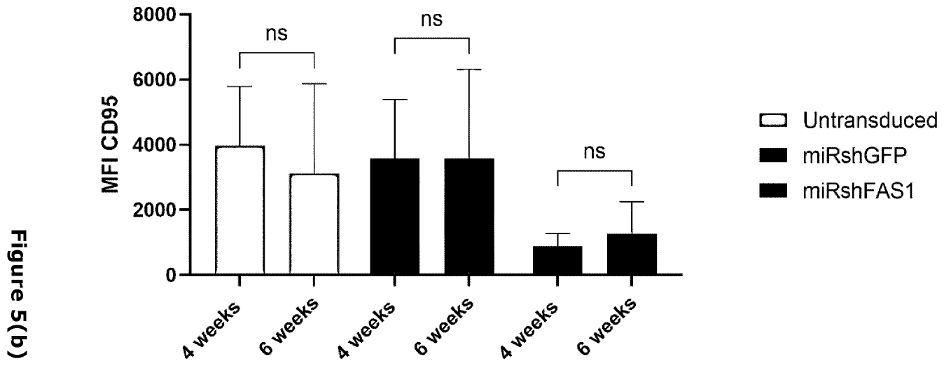

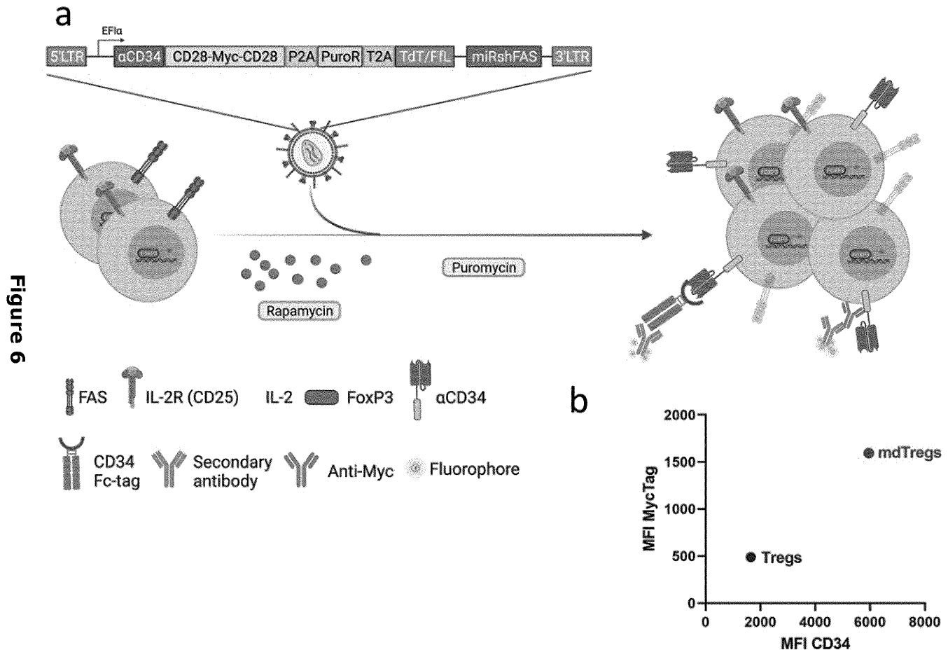

The non-activating CD34-targeting scFv, named 34-m-scFv, comprises an scFv derived from QBEND/10 anti-CD34, a monoclonal antibody specific for CD34 but, crucially, non-activating on binding. Thus, when the scFV binds to its cognate ligand (an antigen of CD34), activation of the downstream signalling pathway of CD34 is reduced or prevented. 34-m-scFv was cloned upstream of the human CD28 hinge and transmembrane domain and is followed in the vector by luciferase for imaging, and the shRNA cassette for FAS KD. Genes were separated by T2A ribosomal skip sequences allowing stoichiometric expression of all components of the vector (FIG. 6A). Expression was driven by an EF1α promoter-modified pUltra lentiviral vector (Addgene #24129) A myc-tag was incorporated in the CD28 hinge for detection purposes. The resultant construct was called 34-m-scFv/FASkd (FIG. 6A).

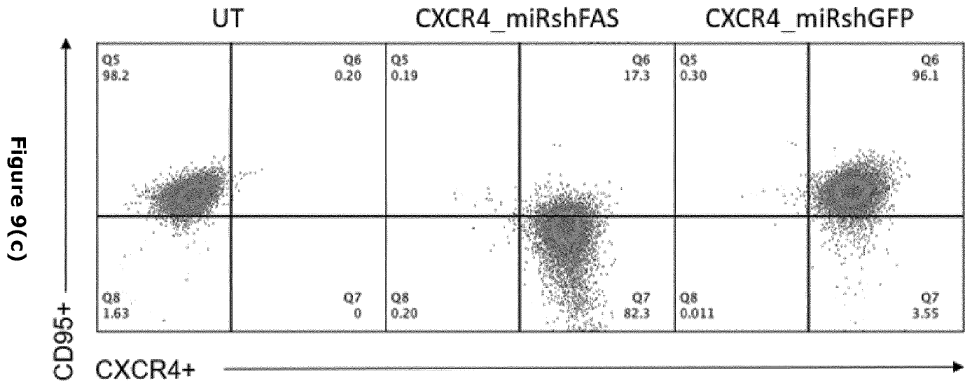

Further genes of interest can be incorporated into the construct with the potential to replace the CD34 targeting or to co-express chemokine receptors, such as CXCR4 (FIG. 9) or a functional variant thereof, to further enhance bone marrow tropism. FIG. 9 shows the successful co-expression of CXCR4 ligand and the miRshFAS1 for the knockdown of FAS (CXCR4_miRshFAS1 construct). This confirms how different genes of interest can be swapped into the construct.

It can be difficult to expand Tregs to sufficient numbers for cell therapy, given their often anergic nature. However, we successfully expanded Tregs by incubating Tregs with anti-CD28/anti-CD3 beads in the presence of IL-2 and Rapamycin. During expansion the Tregs were genetically engineered with the construct of above to successfully express 34-m-scFv and a reduced expression level of FAS. Successful transduction (anti-CD34 expression and a reduced expression level of FAS) was confirmed by two methods. Firstly, the genetically engineered Tregs were incubated with mouse anti-myc (as the reporter) and stained with secondary APC anti-mouse IgG. This acted as a reporter to confirm that the FAS shRNA was successfully expressed. Additionally, the genetically engineered Tregs were incubated with recombinant human CD34-Fc tagged fusion protein (cat 10103-H02H, Sino Biological). The Fc tag allows the detection of CD34 protein, using a secondary anti-Fc antibody (anti-human Alexa Fluor 647). The successful co-expression of Myc (as a marker for the reduced expression level of FAS) and anti-CD34 is shown in FIG. 6B. This confirms that sufficient numbers of Tregs genetically engineered to express a) a non-activating site-targeting receptor or ligand and b) a reduced expression level of FAS can be generated with potential use in cell therapy, particularly in the treatment of aplastic anaemia (AA).

### Example 5: Optimization of the Anti-CD34 scFv Portion of the 34-m-scFv/Faskd Construct

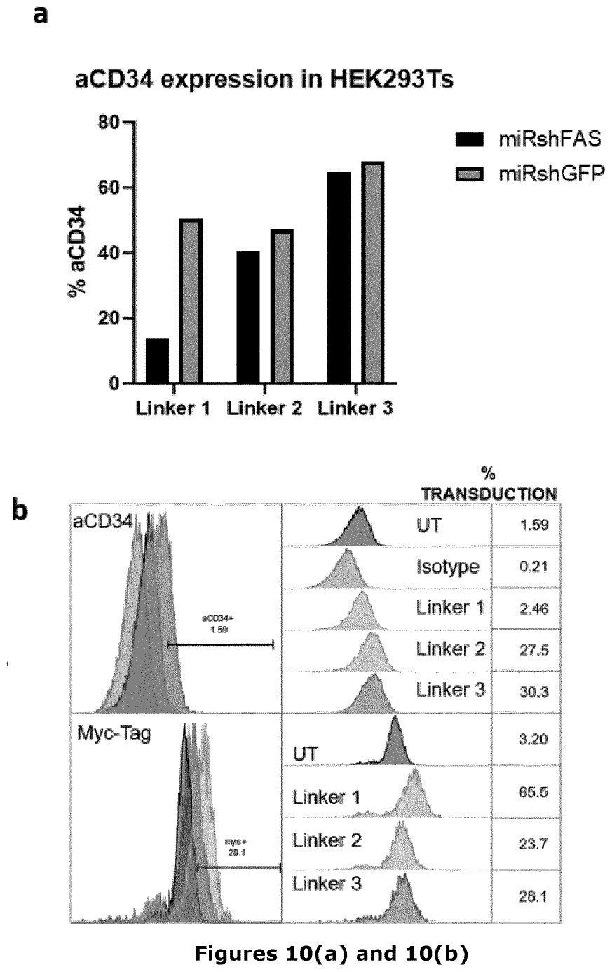

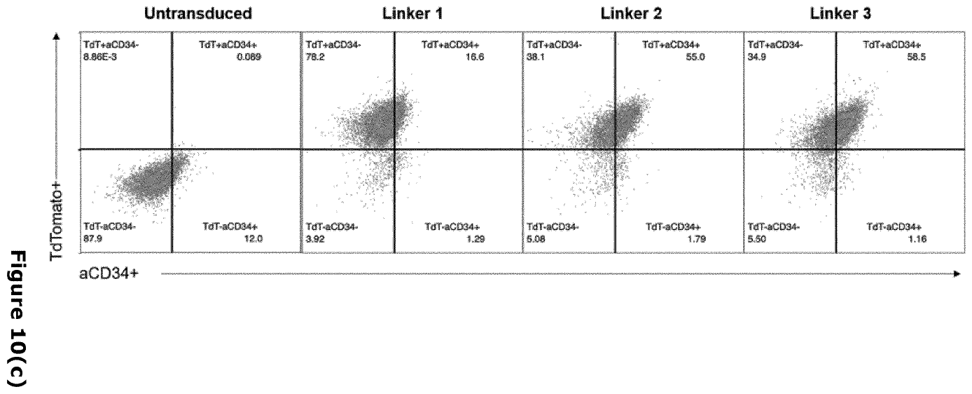

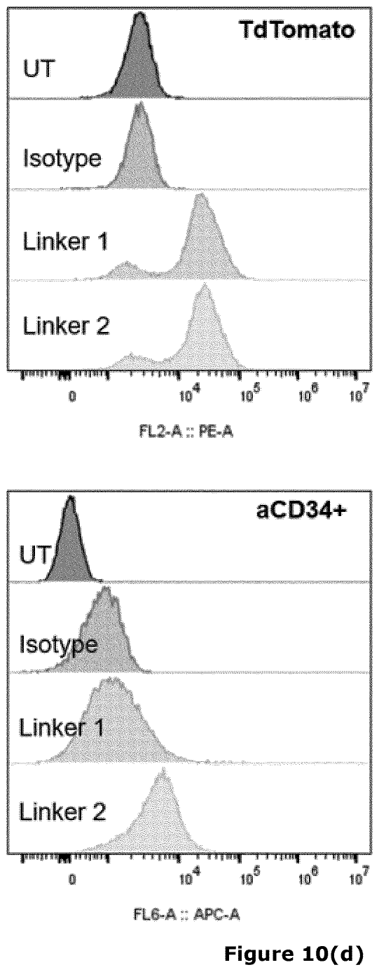

To optimize the binding between the non-activating CD34-targeting scFv of the 34-m-scFv/FASkd construct and its cognate ligand (an antigen of CD34), different linkers were tested for the QBEND/10 anti-CD34 antibody scFv. As before, the expression was subjected by an EF1α promoter-modified pUltra lentiviral vector (Addgene #24129). Thus, four vectors comprising different linker sequences were designed, each containing a different linker sequence (Linker_1 (SEQ ID NO: 31), Linker_2 (SEQ ID NO: 32), Linker_3 (SEQ ID NO: 33), Linker_4 (SEQ ID NO: 34)). As before, TdTomato was used as a reporter gene for the detection of transduced cells. Additionally, a four set of cloning vectors were also designed containing a shRNA targeting GFP as a negative control. FIG. 10 confirms the expression of αCD34 in HEK293T cells (FIG. 10A), Jurkat cells (FIGS. 10B-C) and Tregs (FIG. 10D).

### Example 6: Further Generation of Tregs with Reduced FAS Expression Level

The two FAS-targeting miRNAs comprising shRNAs (miRshFAS1 and miRshFAS2) of Examples 3 and 4 exhibit a good reduction in FAS expression levels. For these miRNAs, the shRNAs were incorporated into the loop structure of the miRNA.

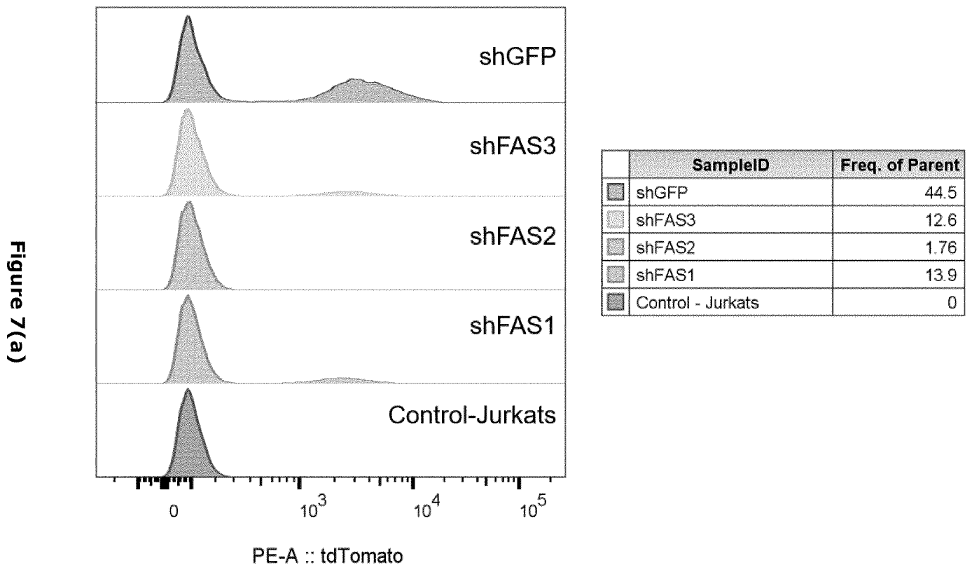

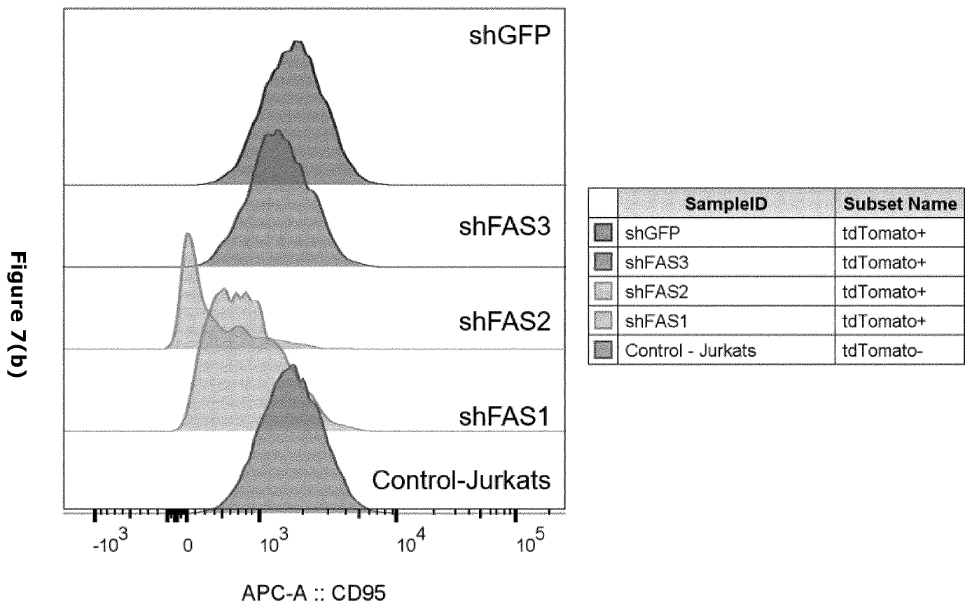

It was decided to test alternative approaches to reduce FAS expression in the Tregs. shRNA sequences targeting FAS were introduced into the Tregs without incorporation into an miRNA/pre/pri-miRNA sequence. Thus, vectors comprising shRNA sequences, but not miRNA sequences, were developed and introduced into the Treg. In particular, three cloning vectors were designed, each containing a different shRNA sequence targeting FAS (shFAS_1 (SEQ ID NO: 1), shFAS_2 (SEQ ID NO: 2) and sh_FAS3 (SEQ ID NO: 30). A further vector was designed containing a shRNA targeting GFP as a negative control. TdTomato was used as a reporter gene for the detection of transduced cells (FIG. 7A). FIG. 7B confirms that shFAS1 and shFAS2 were particularly effective at reducing FAS (CD95) expression.

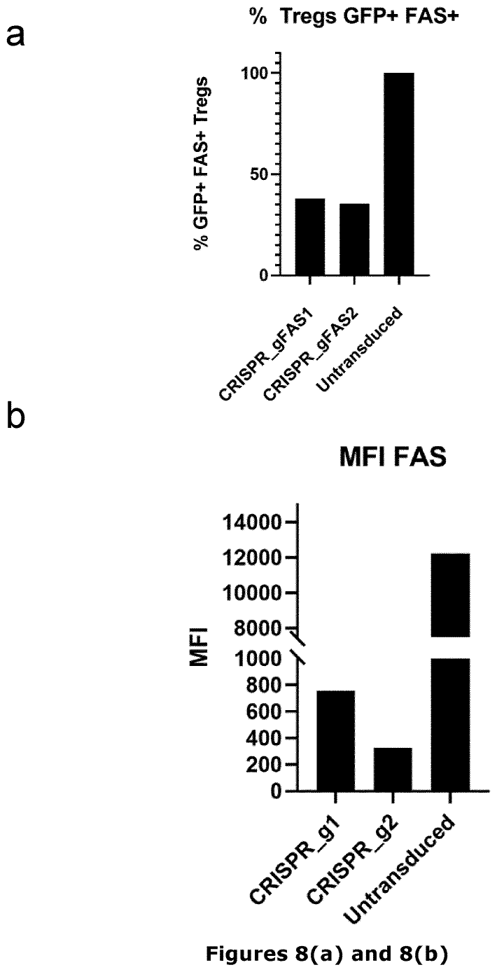

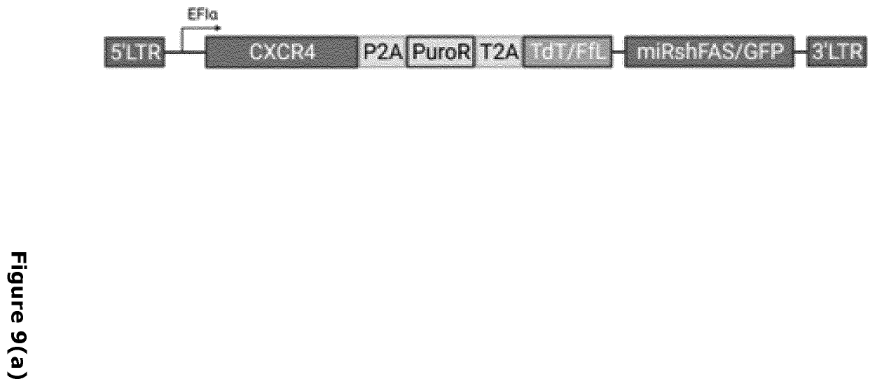

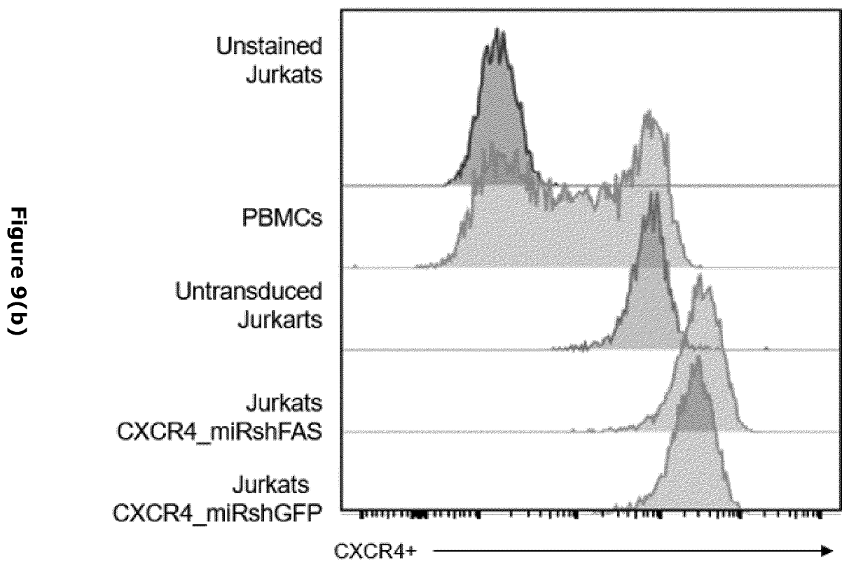

The use of a CRISPR-CAS9 system to reduce FAS expression was then explored. FIG. 8A shows the percentage of cells positive for FAS following the introduction into the cells of two different CRISPR guide RNAs targeting FAS. GFP was used as a reporter gene to detect transduced cells. FIG. 8B shows the FAS MFI in cells following the introduction onto the cells of two different CRISPR guide RNAs targeting FAS. As above, GFP was used as a reporter gene to detect transduced cells. This figure confirms that a CRISPR system can be used to reduce FAS expression in Tregs.

### REFERENCES

Kordasti, S. et al. Deep phenotyping of Tregs identifies an immune signature for idiopathic aplastic anemia and predicts response to treatment. Blood 128, 1193-1205 (2016).
Lim, S. P. et al. Treg sensitivity to FasL and relative IL-2 deprivation drive idiopathic aplastic anemia immune dysfunction. Blood 136, 885-897 (2020).

### SEQUENCES

SEQ ID NO: 1
GCGTATGACACATTGATTAAA
SEQ ID NO: 2
GTGCAGATGTAAACCAAACTT
SEQ ID NO: 3
GCGACUGUAAACAUCCUCGACUGGAAGCUGUGAAGCCACAGAUGGGCUUUCAGUCGGAUGUUUG
CAGCUGC
SEQ ID NO: 4
ACCAAGUUUCAGUUCAUGUAAACAUCCUACACUCAGCUGUAAUACAUGGAUUGGCUGGGAGGUG
GAUGUUUACUUCAGCUGACUUGGA
SEQ ID NO: 5
ACCAUGCUGUAGUGUGUGUAAACAUCCUACACUCUCAGCUGUGAGCUCAAGGUGGCUGGGAGAG
GGUUGUUUACUCCUUCUGCCAUGGA
SEQ ID NO: 6
AGAUACUGUAAACAUCCUACACUCUCAGCUGUGGAAAGUAAGAAAGCUGGGAGAAGGCUGUUUA
CUCUUUCU
SEQ ID NO: 7
GUUGUUGUAAACAUCCCCGACUGGAAGCUGUAAGACACAGCUAAGCUUUCAGUCAGAUGUUUGC
UGCUAC
SEQ ID NO: 8
GGGCAGUCUUUGCUACUGUAAACAUCCUUGACUGGAAGCUGUAAGGUGUUCAGAGGAGCUUUCA
GUCGGAUGUUUACAGCGGCAGGCUGCCA
SEQ ID NO: 9 (miRshFAS1)
tgtttgaatgaggcttcagtactttacagaatcgttgcctgcacatcttggaaacacttgctgggattacttcGACTTCttaacccaa
cagaaggcTCGAGAAGGTATATTGCTGTTGACAGTGAGCGcgcgtatgacacattgattaaaTAGTGAAGCCA
CAGATGTAtttaatcaatgtgtcatacgctTGCCTACTGCCTCGGACTTCAAGGGGCTAGAATTCGAGCAATT
ATCTTGTTTACTAAAACTGAATACCTTGCTATCTCTTTGATACATTTTTACAAAGCTGAATTAAAATGG
TATAAATTAAATCACTTTTTT
SEQ ID NO: 10 (miRshFAS2)
tgtttgaatgaggcttcagtactttacagaatcgttgcctgcacatcttggaaacacttgctgggattacttcGACTTCttaacccaa
cagaaggcTCGAGAAGGTATATTGCTGTTGACAGTGAGCGcgtgcagatgtaaaccaaacttTAGTGAAGCCA
CAGATGTAaagtttggtttacatctgcactTGCCTACTGCCTCGGACTTCAAGGGGCTAGAATTCGAGCAATT
ATCTTGTTTACTAAAACTGAATACCTTGCTATCTCTTTGATACATTTTTACAAAGCTGAATTAAAATGG
TATAAATTAAATCACTTTTTT
SEQ ID NO: 11
QVQLVQSGAELVKPGASVKMSCKASGYTFTSYVIHWVKQKPGQGLEWLGYTNPYNDVTKYNEKFKFKA
TLTSDTSSTTAYMEFSSLTSEDSAVYYCARYGGLWLYAMDYWGQGTSVTVSS
SEQ ID NO: 12
QLVLTQSPSASFSLGASAKLTCTLSSQHRTFTIEWYQQQPEKPPKYVMELRKDGSHSKGDGIPDRFSGS
SSGADRYLSISSIQPEDEAIYICGVGNTIKEQFVYVFGTGTKVTVL
SEQ ID NO: 13
GGGGSGGGGSGGGGS
SEQ ID NO: 14
MALPVTALLLPLALLLHAARPQVQLVQSGAELVKPGASVKMSCKASGYTFTSYVIHWVKQKPGQGLEWL
GYTNPYNDVTKYNEKFKFKATLTSDTSSTTAYMEFSSLTSEDSAVYYCARYGGLWLYAMDYWGQGTSV
TVSS GGGGSGGGGSGGGGS
QLVLTQSPSASFSLGASAKLTCTLSSQHRTFTIEWYQQQPEKPPKYVMELRKDGSHSKGDGIPDRFSGS
SSGADRYLSISSIQPEDEAIYICGVGNTIKEQFVYVFGTGTKVTVL IEV EQKLISEEDL
LDNEKSNGTIIH VKGKHLCPSPLFPGPSKP FWVLVVVGGVLACYSLLVTVAFIIFWV
SEQ ID NO: 15
MALPVTALLLPLALLLHAARPQLVLTQSPSASFSLGASAKLTCTLSSQHRTFTIEWYQQQPEKPPKYVMEL
RKDGSHSKGDGIPDRFSGSSSGADRYLSISSIQPEDEAIYICGVGNTIKEQFVYVFGTGTKVTVLGGGG
SGGGGSGGGGSQVQLVQSGAELVKPGASVKMSCKASGYTFTSYVIHWVKQKPGQGLEWLGYTNPYN
DVTKYNEKFKFKATLTSDTSSTTAYMEFSSLTSEDSAVYYCARYGGLWLYAMDYWGQGTSVTVSSIEV
EQKLISEEDLLDNEKSNGTIIH VKGKHLCPSPLFPGPSKPFWVLVVVGGVLACYSLLVTVAFIIFWV
SEQ ID NO: 16
ATGGCTCTGCCTGTGACAGCTCTGCTGCTGCCTCTGGCTCTGCTGCTGCACGCCGCTAGACCCCAG
GTCCAGCTCGTACAGTCAGGTGCAGAACTCGTCAAACCAGGTGCCTCAGTCAAGATGTCATGCAAAG
CAAGCGGCTACACATTCACTAGTTACGTGATCCACTGGGTCAAGCAGAAACCTGGCCAGGGTCTGG
AGTGGCTCGGCTACACCAACCCTTACAACGACGTGACAAAGTACAACGAAAAGTTCAAGTTCAAGGC
AACTCTGACCTCCGATACGTCGTCTACCACAGCCTACATGGAGTTCAGCTCCCTCACCTCAGAAGAC
AGCGCCGTCTACTATTGCGCTAGATATGGCGGACTGTGGCTCTACGCTATGGATTATTGGGGCCAG
GGAACATCCGTGACTGTCTCTAGTggtggaggTggAtcaggTggaggtggAtctggTggAggTggatctCAGCT
CGTCCTCACTCAGTCTCCCTCAGCCTCCTTTTCCCTCGGTGCCTCCGCCAAACTCACTTGTACTCTCA
GCAGCCAGCATAGAACTTTCACTATCGAGTGGTATCAGCAGCAGCCTGAGAAGCCCCCTAAATATGT
GATGGAACTCCGGAAGGACGGAAGTCACTCAAAGGGTGACGGCATTCCAGATAGGTTTTCTGGTAG
CTCCTCTGGGGCCGACAGATACCTGAGCATCTCCAGCATTCAGCCCGAGGATGAAGCTATCTATATT
TGCGGAGTCGGGAATACCATCAAAGAGCAGTTCGTGTACGTCTTTGGCACAGGGACCAAGGTGACA
GTCCTCgcGGCCGCCATCGAGGTGGAGCAGAAGCTGATCAGCGAGGAGGACCTGCTGGACAACGA
GAAGAGCAACGGCACCATCATCCACGTGAAGGGCAAGCACCTGTGCCCCAGCCCCCTGTTCCCCGG
CCCCAGCAAGCCCTTCTGGGTGCTGGTGGTGGTGGGCGGCGTGCTGGCCTGCTACAGCCTGCTGG
TGACCGTGGCCTTCATCATCTTCTGGGTG
SEQ ID NO: 17
ATGGCTCTGCCTGTGACAGCTCTGCTGCTGCCTCTGGCTCTGCTGCTGCACGCCGCTAGACCCCAGC
TCGTCCTCACTCAGTCTCCCTCAGCCTCCTTTTCCCTCGGTGCCTCCGCCAAACTCACTTGTACTCTC
AGCAGCCAGCATAGAACTTTCACTATCGAGTGGTATCAGCAGCAGCCTGAGAAGCCCCCTAAATATG
TGATGGAACTCCGGAAGGACGGAAGTCACTCAAAGGGTGACGGCATTCCAGATAGGTTTTCTGGTA
GCTCCTCTGGGGCCGACAGATACCTGAGCATCTCCAGCATTCAGCCCGAGGATGAAGCTATCTATAT
TTGCGGAGTCGGGAATACCATCAAAGAGCAGTTCGTGTACGTCTTTGGCACAGGGACCAAGGTGAC
AGTCCTCgcGGCCGCCggtggaggTggAtcaggTggaggtggAtctggTggAggTggatctCAGGTCCAGCTCG
TACAGTCAGGTGCAGAACTCGTCAAACCAGGTGCCTCAGTCAAGATGTCATGCAAAGCAAGCGGCT
ACACATTCACTAGTTACGTGATCCACTGGGTCAAGCAGAAACCTGGCCAGGGTCTGGAGTGGCTCG
GCTACACCAACCCTTACAACGACGTGACAAAGTACAACGAAAAGTTCAAGTTCAAGGCAACTCTGAC
CTCCGATACGTCGTCTACCACAGCCTACATGGAGTTCAGCTCCCTCACCTCAGAAGACAGCGCCGTC
TACTATTGCGCTAGATATGGCGGACTGTGGCTCTACGCTATGGATTATTGGGGCCAGGGAACATCCG
TGACTGTCTCTAGTATCGAGGTGGAGCAGAAGCTGATCAGCGAGGAGGACCTGCTGGACAACGAGA
AGAGCAACGGCACCATCATCCACGTGAAGGGCAAGCACCTGTGCCCCAGCCCCCTGTTCCCCGGCC
CCAGCAAGCCCTTCTGGGTGCTGGTGGTGGTGGGCGGCGTGCTGGCCTGCTACAGCCTGCTGGTG
ACCGTGGCCTTCATCATCTTCTGGGTG
SEQ ID NO: 18
IEVLDNEKSNGTIIH VKGKHLCPSPLFPGPSKP
SEQ ID NO: 19
MALPVTALLLPLALLLHAARP
SEQ ID NO: 20
GTCGTGAAAACTACCCCTAAAAGCTAGCCGCCACCATGACCGAGTACAAGCCCACGG
SEQ ID NO: 21
CCCCTTCCTTCTCCGGATCCGGCACCGGGCTTGCGG
SEQ ID NO: 22
CGAGCTGTACAAGTGATCAGAATTTGTTTGAATGAGGCTTCAGTACTTTAC
SEQ ID NO: 23
TACATCTGTGGCTTCACTATTTAATCAATGTGTCATACGCGCGCTCACTGTCAACAGC
SEQ ID NO: 24
TACATCTGTGGCTTCACTAAAGTTTGGTTTACATCTGCACGCGCTCACTGTCAACAGC
SEQ ID NO: 25
TACATCTGTGGCTTCACTATGAACTTCAGGGTCAGCTTGCTCGCTCACTGTCAACAGC
SEQ ID NO: 26
TAGTGAAGCCACAGATGTATTTAATCAATGTGTCATACGCTTGCCTACTGCCTCGG
SEQ ID NO: 27
TAGTGAAGCCACAGATGTAAAGTTTGGTTTACATCTGCACTTGCCTACTGCCTCGG
SEQ ID NO: 28
TAGTGAAGCCACAGATGTATGAACTTCAGGGTCAGCTTGCCTGCCTACTGCCTCGG
SEQ ID NO: 29
CGACGACTCCGGAACGAATTAAAAAAGTGATITAATTTATACCATTITAATTCAGC
SEQ ID NO: 30
CCTGAAACAGTGGCAATAAA
SEQ ID NO: 31 (LINKER 1)
GCC GAG GCG GCA GCG AAA GAG GCA GCG GCC AAA GCA
SEQ ID NO: 32 (LINKER 2)
GGT GGA GGC GGG TCT GGA GGA GGA GGA TCA GGC GGG GGA GGA TCT GGT GGG GGC G
GA TCA GGG GGG GGA GGG TCT
SEQ ID NO: 33 (LINKER 3)
GGT GGA GGC GGG TCT GGA GGA GGA GGA TCA GGC GGG GGA GGA TCT GGT GGG GGC G
GA TCA GGG GGG GGA GGG TCT GGC GGA GGT GGC TCT
SEQ ID NO: 34 (LINKER 4)
GGTGGG GGA GGG TCA GGA GGT GGT GGG AGC GGA GGT GGT GGC AGT GGC GGA GGT G
GC TCT
SEQ ID NO: 35 (CXCR4)
ATGGAAGGAATAAGCATTTACACATCAGATAACTACACGGAAGAAATGGGCTCCGGGGATTACGATA
GCATGAAGGAACCATGTTTTCGGGAAGAAAATGCAAACTTTAATAAGATCTTCCTGCCAACCATCTAT
AGTATCATATTTCTGACTGGAATAGTGGGAAACGGTTTGGTAATTCTTGTCATGGGCTATCAAAAGAA
GTTGAGGTCCATGACGGACAAATACCGACTTCATCTCTCTGTAGCCGATCTTCTCTTTGTTATCACCT
TGCCCTTCTGGGCTGTTGATGCGGTGGCTAATTGGTACTTCGGTAACTTCCTCTGCAAAGCCGTGCA
TGTCATCTACACAGTAAACTTGTATTCTAGCGTCTTGATCCTTGCCTTCATCTCTCTTGATAGGTATTT
GGCTATAGTACATGCGACCAACTCCCAACGCCCCCGAAAACTCCTCGCCGAAAAAGTCGTATATGTG
GGTGTTTGGATACCTGCCCTCCTGCTCACTATACCAGATTTCATATTTGCAAATGTGTCAGAAGCGGA
CGATCGGTATATATGCGACAGATTTTATCCGAACGACCTCTGGGTTGTAGTCTTTCAATTCCAACACA
TCATGGTAGGACTGATATTGCCGGGCATTGTAATTTTGTCATGTTACTGCATAATTATAAGTAAACTC
TCTCACTCTAAAGGTCATCAAAAACGCAAGGCACTCAAGACCACGGTGATCCTGATCTTGGCGTTCT
TCGCTTGCTGGCTGCCATATTATATAGGAATTAGCATTGATTCATTTATACTTCTCGAAATCATAAAAC
AGGGATGCGAGTTTGAGAATACTGTGCATAAGTGGATCAGTATTACGGAGGCTCTTGCATTCTTTCA
CTGTTGCCTCAACCCTATCCTCTACGCGTTCCTCGGGGCAAAATTCAAAACTTCCGCACAGCACGCC
TTGACAAGCGTGAGCAGGGGATCTTCCTTGAAGATTTTGAGCAAGGGCAAGCGGGGGGGACACTCC
AGTGTGAGTACGGAATCTGAGTCCTCATCTTTTCATTCTAGC

1. A regulatory T-cell (Treg) genetically engineered to express:
a) a non-activating site-targeting receptor or ligand; and
b) a reduced expression level of FAS.

2. The Treg according to claim 1, wherein the Treg is genetically engineered to express a non-activating site-targeting receptor.

3. The Treg according to claim 1 or claim 2, wherein the expression level of FAS is reduced by at least about 50%

4. The Treg according to any one of claims 1 to 3, wherein the Treg is genetically engineered to express a nucleotide sequence which reduces the expression level of FAS.

5. The Treg according to claim 4, wherein the nucleotide sequence comprises RNA.

6. The Treg according to claim 4 or claim 5, wherein the nucleotide sequence comprises short hairpin RNA (shRNA), an antisense oligonucleotide, double stranded RNA (dsRNA), or a CRISPR guide RNA.

7. The Treg according to claim 5 or claim 6, wherein the nucleotide sequence comprises shRNA.

8. The Treg according to claim 7, wherein the nucleotide sequence comprises a microRNA (miRNA), pre-miRNA and/or pri-miRNA comprising a shRNA.

9. The Treg according to claim 7 or claim 8, wherein the shRNA is encoded by SEQ ID NO: 1, SEQ ID NO: 2 or a functional variant thereof.

10. The Treg according to any one of claims 4 to 9, wherein the Treg comprises a vector encoding the nucleotide sequence, optionally wherein the vector further comprises a nucleotide sequence encoding the non-activating site-targeting receptor or ligand.

11. The Treg according to any one of the preceding claims, wherein the site comprises one or more of bone marrow, tumour microenvironment (TME), central nervous system (CNS), lungs, liver, bowel, bladder, large intestine, small intestine and stomach.

12. The Treg according to any one of the preceding claims, wherein the site comprises bone marrow.

13. The Treg according to any one of the preceding claims, wherein the non-activating site-targeting receptor or ligand does not comprise an intracellular domain.

14. The Treg according to any one of the preceding claims, wherein the non-activating site-targeting receptor comprises a non-activating site-targeting antibody or variant thereof.

15. The Treg according to claim 14, wherein the non-activating site-targeting antibody or variant thereof is a non-activating anti-CD34 antibody or variant thereof.

16. The Treg according to claim 15, wherein the non-activating site-targeting antibody or variant thereof is a non-activating site targeting antibody variant comprising a non-activating anti-CD34 scFv.

17. The Treg according to any one of the preceding claims, wherein the Treg is genetically engineered to further express:
c) a chemokine receptor or a functional variant thereof.

18. The Treg according to claim 17, wherein the chemokine receptor comprises CXCR4 or a functional variant thereof.

19. An or a plurality of polynucleotide sequence(s) comprising:
a) a nucleotide sequence which reduces the expression level of FAS; and
b) a nucleotide sequence encoding a non-activating site-targeting receptor or ligand; and optionally
c) a nucleotide sequence encoding a chemokine receptor or a functional variant thereof.

20. A vector comprising the polynucleotide sequence(s) according to claim 19.

21. A method of modifying a Treg, the method comprising genetically engineering a Treg to express:
a) a non-activating site-targeting receptor or ligand; and
b) a reduced expression level of FAS.

22. A pharmaceutical composition comprising the Treg according to any one of claims 1-18, the polynucleotide sequence(s) according to claim 19 and/or the vector according to claim 20 and a pharmaceutically or physiologically acceptable diluent and/or carrier.

23. The Treg according to any one of claims 1-18, the polynucleotide sequence(s) according to claim 19, the vector according to claim 20 or the pharmaceutical composition according to claim 22 for use in the treatment or prevention of a disease.

24. A method of treating or preventing a disease in a subject, wherein the method comprises administering to the subject the Treg according to any one of claims 1-18, the polynucleotide sequence(s) according to claim 19, the vector according to claim 20 or the pharmaceutical composition according to claim 22.

25. The Treg, polynucleotide sequence(s), vector or pharmaceutical composition for use of claim 23 or the method of claim 24, wherein the disease comprises an autoimmune disease.

26. The Treg, polynucleotide sequence(s), vector or pharmaceutical composition for use of claim 23 or claim 25, or the method of claim 24 or claim 25, wherein the disease comprises Aplastic Anaemia (AA).


SEQUENCE LISTING (30 sequences)

SEQ ID NO: 1 - DNA, 21 bp, synthetic construct
  source 1..21: mol_type = other DNA
gcgtatgacacattgattaaa

SEQ ID NO: 2 - DNA, 21 bp, synthetic construct
  source 1..21: mol_type = other DNA
gtgcagatgtaaaccaaactt

SEQ ID NO: 3 - RNA, 71 nt, Homo sapiens
  source 1..71: mol_type = other RNA
gcgactgtaaacatcctcgactggaagctgtgaagccacagatgggctttcagtcggatgtttgcagctgc

SEQ ID NO: 4 - RNA, 88 nt, Homo sapiens
  source 1..88: mol_type = other RNA
accaagtttcagttcatgtaaacatcctacactcagctgtaatacatggattggctgggaggtggatgtttacttcagctgacttgga

SEQ ID NO: 5 - RNA, 89 nt, Homo sapiens
  source 1..89: mol_type = other RNA
accatgctgtagtgtgtgtaaacatcctacactctcagctgtgagctcaaggtggctgggagagggttgtttactccttctgccatgga

SEQ ID NO: 6 - RNA, 72 nt, Homo sapiens
  source 1..72: mol_type = other RNA
agatactgtaaacatcctacactctcagctgtggaaagtaagaaagctgggagaaggctgtttactctttct

SEQ ID NO: 7 - RNA, 70 nt, Homo sapiens
  source 1..70: mol_type = other RNA
gttgttgtaaacatccccgactggaagctgtaagacacagctaagctttcagtcagatgtttgctgctac

SEQ ID NO: 8 - RNA, 92 nt, Homo sapiens
  source 1..92: mol_type = other RNA
gggcagtctttgctactgtaaacatccttgactggaagctgtaaggtgttcagaggagctttcagtcggatgtttacagcggcaggctgcca

SEQ ID NO: 9 - DNA, 324 bp, synthetic construct
  source 1..324: mol_type = other DNA
tgtttgaatgaggcttcagtactttacagaatcgttgcctgcacatcttggaaacacttgctgggattacttcgacttcttaacccaacagaaggctcgagaaggtatattgctgttgacagtgagcgcgcgtatgacacattgattaaatagtgaagccacagatgtatttaatcaatgtgtcatacgcttgcctactgcctcggacttcaaggggctagaattcgagcaattatcttgtttactaaaactgaataccttgctatctctttgatacatttttacaaagctgaattaaaatggtataaattaaatcactttttt

SEQ ID NO: 10 - DNA, 324 bp, synthetic construct
  source 1..324: mol_type = other DNA
tgtttgaatgaggcttcagtactttacagaatcgttgcctgcacatcttggaaacacttgctgggattacttcgacttcttaacccaacagaaggctcgagaaggtatattgctgttgacagtgagcgcgtgcagatgtaaaccaaactttagtgaagccacagatgtaaagtttggtttacatctgcacttgcctactgcctcggacttcaaggggctagaattcgagcaattatcttgtttactaaaactgaataccttgctatctctttgatacatttttacaaagctgaattaaaatggtataaattaaatcactttttt

SEQ ID NO: 11 - protein, 120 aa, synthetic construct
QVQLVQSGAELVKPGASVKMSCKASGYTFTSYVIHWVKQKPGQGLEWLGYTNPYNDVTKYNEKFKFKATLTSDTSSTTAYMEFSSLTSEDSAVYYCARYGGLWLYAMDYWGQGTSVTVSS

SEQ ID NO: 12 - protein, 115 aa, synthetic construct
QLVLTQSPSASFSLGASAKLTCTLSSQHRTFTIEWYQQQPEKPPKYVMELRKDGSHSKGDGIPDRFSGSSSGADRYLSISSIQPEDEAIYICGVGNTIKEQFVYVFGTGTKVTVL

SEQ ID NO: 13 - protein, 15 aa, synthetic construct
GGGGSGGGGSGGGGS

SEQ ID NO: 14 - protein, 341 aa, synthetic construct
MALPVTALLLPLALLLHAARPQVQLVQSGAELVKPGASVKMSCKASGYTFTSYVIHWVKQKPGQGLEWLGYTNPYNDVTKYNEKFKFKATLTSDTSSTTAYMEFSSLTSEDSAVYYCARYGGLWLYAMDYWGQGTSVTVSSGGGGSGGGGSGGGGSQLVLTQSPSASFSLGASAKLTCTLSSQHRTFTIEWYQQQPEKPPKYVMELRKDGSHSKGDGIPDRFSGSSSGADRYLSISSIQPEDEAIYICGVGNTIKEQFVYVFGTGTKVTVLIEVEQKLISEEDLLDNEKSNGTIIHVKGKHLCPSPLFPGPSKPFWVLVVVGGVLACYSLLVTVAFIIFWV

SEQ ID NO: 15 - protein, 341 aa, synthetic construct
MALPVTALLLPLALLLHAARPQLVLTQSPSASFSLGASAKLTCTLSSQHRTFTIEWYQQQPEKPPKYVMELRKDGSHSKGDGIPDRFSGSSSGADRYLSISSIQPEDEAIYICGVGNTIKEQFVYVFGTGTKVTVLGGGGSGGGGSGGGGSQVQLVQSGAELVKPGASVKMSCKASGYTFTSYVIHWVKQKPGQGLEWLGYTNPYNDVTKYNEKFKFKATLTSDTSSTTAYMEFSSLTSEDSAVYYCARYGGLWLYAMDYWGQGTSVTVSSIEVEQKLISEEDLLDNEKSNGTIIHVKGKHLCPSPLFPGPSKPFWVLVVVGGVLACYSLLVTVAFIIFWV

SEQ ID NO: 16 - DNA, 1032 bp, synthetic construct
  source 1..1032: mol_type = other DNA
atggctctgcctgtgacagctctgctgctgcctctggctctgctgctgcacgccgctagaccccaggtccagctcgtacagtcaggtgcagaactcgtcaaaccaggtgcctcagtcaagatgtcatgcaaagcaagcggctacacattcactagttacgtgatccactgggtcaagcagaaacctggccagggtctggagtggctcggctacaccaacccttacaacgacgtgacaaagtacaacgaaaagttcaagttcaaggcaactctgacctccgatacgtcgtctaccacagcctacatggagttcagctccctcacctcagaagacagcgccgtctactattgcgctagatatggcggactgtggctctacgctatggattattggggccagggaacatccgtgactgtctctagtggtggaggtggatcaggtggaggtggatctggtggaggtggatctcagctcgtcctcactcagtctccctcagcctccttttccctcggtgcctccgccaaactcacttgtactctcagcagccagcatagaactttcactatcgagtggtatcagcagcagcctgagaagccccctaaatatgtgatggaactccggaaggacggaagtcactcaaagggtgacggcattccagataggttttctggtagctcctctggggccgacagatacctgagcatctccagcattcagcccgaggatgaagctatctatatttgcggagtcgggaataccatcaaagagcagttcgtgtacgtctttggcacagggaccaaggtgacagtcctcgcggccgccatcgaggtggagcagaagctgatcagcgaggaggacctgctggacaacgagaagagcaacggcaccatcatccacgtgaagggcaagcacctgtgccccagccccctgttccccggccccagcaagcccttctgggtgctggtggtggtgggcggcgtgctggcctgctacagcctgctggtgaccgtggccttcatcatcttctgggtg

SEQ ID NO: 17 - DNA, 1032 bp, synthetic construct
  source 1..1032: mol_type = other DNA
atggctctgcctgtgacagctctgctgctgcctctggctctgctgctgcacgccgctagaccccagctcgtcctcactcagtctccctcagcctccttttccctcggtgcctccgccaaactcacttgtactctcagcagccagcatagaactttcactatcgagtggtatcagcagcagcctgagaagccccctaaatatgtgatggaactccggaaggacggaagtcactcaaagggtgacggcattccagataggttttctggtagctcctctggggccgacagatacctgagcatctccagcattcagcccgaggatgaagctatctatatttgcggagtcgggaataccatcaaagagcagttcgtgtacgtctttggcacagggaccaaggtgacagtcctcgcggccgccggtggaggtggatcaggtggaggtggatctggtggaggtggatctcaggtccagctcgtacagtcaggtgcagaactcgtcaaaccaggtgcctcagtcaagatgtcatgcaaagcaagcggctacacattcactagttacgtgatccactgggtcaagcagaaacctggccagggtctggagtggctcggctacaccaacccttacaacgacgtgacaaagtacaacgaaaagttcaagttcaaggcaactctgacctccgatacgtcgtctaccacagcctacatggagttcagctccctcacctcagaagacagcgccgtctactattgcgctagatatggcggactgtggctctacgctatggattattggggccagggaacatccgtgactgtctctagtatcgaggtggagcagaagctgatcagcgaggaggacctgctggacaacgagaagagcaacggcaccatcatccacgtgaagggcaagcacctgtgccccagccccctgttccccggccccagcaagcccttctgggtgctggtggtggtgggcggcgtgctggcctgctacagcctgctggtgaccgtggccttcatcatcttctgggtg

SEQ ID NO: 18 - protein, 33 aa, Homo sapiens
IEVLDNEKSNGTIIHVKGKHLCPSPLFPGPSKP

SEQ ID NO: 19 - protein, 21 aa, Homo sapiens
MALPVTALLLPLALLLHAARP

SEQ ID NO: 20 - DNA, 57 bp, synthetic construct
  source 1..57: mol_type = other DNA
gtcgtgaaaactacccctaaaagctagccgccaccatgaccgagtacaagcccacgg

SEQ ID NO: 21 - DNA, 36 bp, synthetic construct
  source 1..36: mol_type = other DNA
ccccttccttctccggatccggcaccgggcttgcgg

SEQ ID NO: 22 - DNA, 51 bp, synthetic construct
  source 1..51: mol_type = other DNA
cgagctgtacaagtgatcagaatttgtttgaatgaggcttcagtactttac

SEQ ID NO: 23 - DNA, 58 bp, synthetic construct
  source 1..58: mol_type = other DNA
tacatctgtggcttcactatttaatcaatgtgtcatacgcgcgctcactgtcaacagc

SEQ ID NO: 24 - DNA, 58 bp, synthetic construct
  source 1..58: mol_type = other DNA
tacatctgtggcttcactaaagtttggtttacatctgcacgcgctcactgtcaacagc

SEQ ID NO: 25 - DNA, 58 bp, synthetic construct
  source 1..58: mol_type = other DNA
tacatctgtggcttcactatgaacttcagggtcagcttgctcgctcactgtcaacagc

SEQ ID NO: 26 - DNA, 56 bp, synthetic construct
  source 1..56: mol_type = other DNA
tagtgaagccacagatgtatttaatcaatgtgtcatacgcttgcctactgcctcgg

SEQ ID NO: 27 - DNA, 56 bp, synthetic construct
  source 1..56: mol_type = other DNA
tagtgaagccacagatgtaaagtttggtttacatctgcacttgcctactgcctcgg

SEQ ID NO: 28 - DNA, 56 bp, synthetic construct
  source 1..56: mol_type = other DNA
tagtgaagccacagatgtatgaacttcagggtcagcttgcctgcctactgcctcgg

SEQ ID NO: 29 - DNA, 56 bp, synthetic construct
  source 1..56: mol_type = other DNA
cgacgactccggaacgaattaaaaaagtgatttaatttataccattttaattcagc

SEQ ID NO: 30 - DNA, 20 bp, synthetic construct
  source 1..20: mol_type = other DNA
cctgaaacagtggcaataaa